(19) United States
(12) Patent Application Publication
(10) Pub. No.: US 2020/0263187 A1
(43) Pub. Date: Aug. 20, 2020

(54) A METHOD FOR EXPRESSION OF A PROKARYOTIC MEMBRANE PROTEIN IN AN EUKARYOTIC ORGANISM, PRODUCTS AND USES THEREOF

(71) Applicant: Universidade Do Minho, Braga (PT)

(72) Inventors: Margarida Paula PEDRA AMORIM CASAL, Braga (PT); Isabel João SOARES DA SILVA, Braga (PT); Sandra Cristina ALMEIDA PAIVA, Braga (PT); David Manuel NOGUEIRA RIBAS, Braga (PT); Ana Carolina GOMES ALMEIDA, Braga (PT)

(21) Appl. No.: 16/651,982

(22) PCT Filed: Sep. 28, 2018
(86) PCT No.: PCT/IB2018/057572

§ 371 Date: Mar. 27, 2020

(30) Foreign Application Priority Data
Sep. 28, 2017 (PT) 110312

(51) Int. Cl. C12N 15/81; C07K 14/395; C12N 9/12

(52) U.S. Cl. CPC C12N 15/81; C12N 9/1205; C07K 14/395

(57) ABSTRACT

A method for the production of a functional prokaryotic membrane transporter protein in a eukaryotic host organism comprising the following steps: obtaining a DNA construct by ligating a DNA coding sequence of a prokaryotic membrane transporter protein to the N-terminal and/or C-terminal DNA coding sequences of a eukaryotic membrane protein; introducing the obtained DNA construct in the eukaryotic host organism for the production of the functional prokaryotic membrane transporter protein.

### TECHNICAL FIELD

The present disclosure relates to a heterologous expression system to functionally express prokaryotic membrane transporter proteins in eukaryotic organisms. More specifically the disclosure comprises the genetic engineering of chimeric proteins through the combination of a prokaryotic membrane transporter protein sequence with the N-terminus or/and C-terminus coding sequences of a eukaryotic membrane protein and subsequently the efficient functional expression of this genetic engineered chimeric protein into a eukaryotic host.

Surprisingly the method of the present disclosure has the ability to overcome a major bottleneck existing in the biotechnological industry, by allowing the successful functional expression of functional prokaryotic membrane transporter in eukaryotic cells. The impact of the present disclosure is translated in an increased range of substrates able to efficiently permeate the cell membrane of eukaryotic organisms envisaging biotechnological applications, such as substrates previously known not to be transported by the host organism and/or to improve the existing transport properties in terms of kinetics, energetics, import and export capacity and specificity.

### BACKGROUND

The heterologous expression of membrane proteins in host organisms is used since the 1980s. From a biotechnological point of view, the heterologous expression of membrane proteins, such as transporters, allows the host cell to permeate a particular molecule that is unable to cross the cell membrane, or to improve the transport capacity of a particular molecule if the existent cell host transporters are not efficient enough. Other applications, such as functional and structural characterization of membrane proteins are also embraced by this expression system (see review Haferkamp & Linka, 2012; Frommer and Ninneman, 1995). There is a vast list of experiments reporting functional expression of eukaryotic membrane proteins in prokaryotic organisms, namely in Escherichia coli (see review Haferkamp & Linka, 2012). In 1978, a novel method for yeast transformation was developed, enabling the development of new approaches in molecular biology, namely to isolate and characterize eukaryotic genes (Hinnen, Hicks and Fink 1978). The subsequent emergence of new vectors able to replicate both in yeast and bacteria, known as shuttle vectors, was one of such breakthroughs. One of these vectors allowed to revert the leucine auxotrophy in yeast leu2 strain, by transformation with E. coli genomic material (Beggs 1978). A year later, the arginine permease of S. cerevisiae was isolated, using the double mutant leu2/canl (Broach, Strathern and Hicks 1979).

Since 1986, yeast cells were used to heterologously express membrane proteins from other eukaryotic organisms. Interestingly, yeast organisms revealed to be very successful model systems for the expression of plant membrane proteins (Fujita, et al. 1986).

In eukaryotes, the correct delivery of membrane proteins to the endoplasmic reticulum is crucial to later assure the right functionality of these biomolecules at the cell membrane (Cross, et al. 2009). This process can be achieved either by a post-translational modification pathway, involving ATP-binding factors and chaperones after the polypeptides being completed, or by a co-translational pathway GTP-dependent, which occurs during protein synthesis. From an evolutionary point of view, it is thought that co-translational pathway evolved after the post-translational delivery. The co-translational delivery overcomes several problems faced during post-translational process, namely those that comprise the synthesis of complex folding domains, as well as better suits the delivery of membrane proteins. During the integration of protein into membranes, the delivery pathway taken by each protein is strongly affected by the presence and location of specific signal sequences in the newly synthesized polypeptide. Such sequences are composed of a span of hydrophobic amino acid residues. In secretory proteins, this signal sequence is usually located in the protein N-terminal and is cleaved once the protein has crossed the membrane (Cross, et al. 2009). In membrane proteins, similar cleavable N-terminal signals exist or in alternative the hydrophobic transmembrane-spanning region is responsible for directing these proteins to the membrane. The role of the hydrophobic signal sequence in directing proteins to the membrane is clearly conserved between prokaryotes and eukaryotes, although the precise composition of such sequences varies widely (for a review see Cross et al., 2009).

One of the most significant differences between prokaryotic and eukaryotic transporters is the N and C termini length. While in prokaryotic organisms, the N and C terminals are quite short and in most cases almost inexistent, eukaryotic transporters have noticeable bigger terminal domains. It was argued that the unsuccessful expression of some prokaryotic membrane protein, such as the xylose transporter encoded by XylE from E. coli, in S. cerevisiae could be due to membrane incompatibility, low expression levels, and folding difficulties experienced with bacterial proteins (Young, et al. 2011).

The experiments used to validate the present intellectual property will involve two eukaryotic transporters, ScJen1 (lactate transporter) and Hxt1 (glucose transporter), as well as three prokaryotic transporters, namely LIdP (lactate transporter), LctP (lactate transporter) and XylE (xylose transporter).

The ScJen1p was the first monocarboxylic acid transporter described in fungi (Casal, et al. 1999). Besides its role in the uptake of lactate, pyruvate, acetate and propionate (Casal, et al. 1999), it also transports the micronutrient selenite (McDermott, Rosen and Liu 2010) and the antitumor compound 3-bromopyruvate (Lis, et al. 2012). Jen1 has the common topology of the MFS members, known as MFS fold, which comprises 12 TMS (TransMembrane segment) organized in 6+6 folded domains close to the N- and C-termini, separated by a central cytoplasmic loop (Casal, et al. 2016). The transport of the substrate is bidirectional, being Jen1 also involved in the efflux of its substrates (Pacheco, et al. 2012, van Maris, et al. 2004). In S. cerevisiae W303-1A lactic acid-grown cells the estimated kinetic parameters for lactate uptake are: Vmax of 0.40 nmol of lactic acid s1 mg of dry weight1 and Km of 0.29 mM lactic acid (Casal, et al. 1999, Paiva, et al. 2013). In lactic acid, pyruvic acid, acetic acid or glycerol-grown cells JEN1 is highly expressed, whereas in glucose, formic and propionic acid-grown cells it is undetectable (Casal, et al. 1999). Another level of Jen1 regulation involves protein traffic and turnover. The addition of a pulse of glucose to lactic acid-grown cells rapidly triggers the loss of Jen1 activity and endocytosis, followed by vacuolar degradation (Paiva, Kruckeberg and Casal 2002).

The Hxt1 transporter is known as a low affinity glucose transporter (Özcan and Johnston 1999). Hxt1 is a member of the Sugar Porter Family that belongs to the MFS and has a topology of 12 TMS according to the TCDB (2.A.1.1.108). The HXT1 gene expression increases linearly with increasing concentrations of external glucose and achieves full induction at 4% glucose (Özcan and Johnston 1999). The Hxt1p is responsible for the transport of glucose and mannose, by a facilitated-diffusion mechanism (Maier, et al. 2002). The expression of HXT1 in the hxt null mutant EBY.4000 strain (Wieczorke, et al. 1999) restores growth only on high concentrations of glucose, above 1%, and provides low-affinity glucose transport with a Km of 100 mM (Özcan and Johnston 1999).

In E. coli there are two D-lactate transporters characterized, GIcA and LIdP, however mutant analysis proved that the LIdP permease is the main responsible for lactate uptake (Núñez, et al. 2001). According to the Transport Classification Database (TCDB—www.tcdb.org), the E. coli lactate permease LIdP belongs to the Lactate Permease (LctP) family and comprises 12 TMS. Núñez and co-workers (2001) reported LIdP as a permease for glycolate, L-lactate and D-lactate. Another homologue of LIdP transporter is the LctP from Staphylococcus aureus a putative lactate permease also with 12 TMS (Dobson, Reményi and Tusnády 2015).

The XylE transporter from E. coli is known to transport xylose, and binds glucose and 6-bromo-6-deoxy-D-glucose (Sun, et al. 2012). The XylE is also a member of the Sugar Porter Family that belongs to the MFS and has a topology of 12 TMS (TCDB 2.A.1.1.3). XylE is a D-xylose/proton symporter, one of two systems in E. coli K-12 responsible for the uptake of D-xylose (Davis and Henderson 1987).

The 3D structure is known in three conformers, outward occluded, inward occluded and inward open and several substrate-binding residues are conserved with the human Glut-1, 2, 3 and 4 homologues (Quistgaard, et al. 2013).

These facts are disclosed in order to illustrate the technical problem addressed by the present disclosure.

### General Description

The present disclosure comprises the construction of a heterologous expression system, which is based in the genetic fusion of N or/and C terminals coding DNA sequences of eukaryote membrane proteins with the DNA coding sequences of prokaryotic membrane transporter proteins at the beginning and end of the protein DNA sequence, respectively, originating a protein chimera (FIG. 1). This genetic construct is inserted in an expression vector adequate for the expression in the desired host eukaryotic organism.

One of the aims of the present disclosure is to provide a heterologous eukaryote expression system that allows to express a wide range of membrane proteins already characterized and described in prokaryotes or putative transporter proteins.

Another aim of the present disclosure is to deliver chimeric membrane proteins that can increase the range of compounds transported by a particular eukaryote host organism.

Another aim of the present disclosure is to provide chimeric membrane proteins able to increase the transport capacity of certain substrates.

Another aim of the present disclosure is to create chimeric membrane proteins to increase cell factories productivity by increasing the import of molecules/substrates or the export of bio-products.

Another aim of the present disclosure is to provide chimeric membrane proteins able to increase the tolerance of eukaryotic organisms to intracellular compounds through the export of these molecules.

Another aim of the present disclosure is to take advantage of eukaryotic cell properties to favour the functional characterization of prokaryotic membrane transporter proteins.

An aspect of the present disclosure relates to a method for the production of a functional prokaryotic transporter membrane transporter protein in a eukaryotic host organism comprising the following steps:
obtaining a DNA construct by ligating/fusing a DNA coding sequence of a prokaryotic transporter membrane transporter protein to the N-terminal and/or C-terminal DNA coding sequences of a eukaryotic membrane protein; i.e. from the initial codon until the DNA sequence that codes for the first predicted transmembrane segment of a eukaryote membrane protein; introducing the obtained DNA construct in the eukaryotic host organism for the production of the functional prokaryotic membrane transporter protein.

It is considered that, a functional transporter protein is able to transport substrate(s) from one side of a biological membrane to the other, being the type of substrate(s) and transport mechanism defined by the protein sequence. Protein functionality may be evaluated by growth test, uptake/export of radiolabelled substrates, resistance to toxic compounds, etc. depending on the type of protein expressed.

In an embodiment for better results, the DNA construct is obtained by ligating the DNA coding sequence for the prokaryotic membrane transporter protein between the N-terminal and the C-terminal DNA coding sequences of the eukaryotic membrane protein. In particular, are preferred the preferred portions of the sequence, which code for one or more parts of the N-terminal domain of the adenylyl cyclase. The N-terminal domain of the adenylyl cyclase comprises six transmembrane spans, which are especially suited in order to target the membrane protein of interest to the membrane in the expression system. According to the disclosure sequences are used which code for one or more of the transmembrane spans or parts thereof.

In an embodiment for better results, the N-terminal coding DNA sequence is ligated before the initiation codon of the DNA coding sequence for the prokaryotic protein, and the C-terminal coding sequence is ligated after the penultimate codon of the DNA coding sequence for the prokaryotic protein.

In an embodiment for better results, the eukaryotic organism is a fungus; in particular a yeast, more in particular S. cerevisiae.

In an embodiment for better results, the DNA coding sequence for the prokaryotic membrane transporter protein is from is a bacterium, in particular a gram, more in particular a more in particular bacterium without high lipid and mycolic acid content in its cell wall, even more in particular a E. coli, S. aureus, or combinations thereof.

In an embodiment for better results, the eukaryotic membrane protein is a membrane transporter protein.

In an embodiment for better results, the prokaryotic membrane transporter protein is a permease, in particular an organic acid permease, a sugar permease, or mixture thereof.

In an embodiment for better results, the membrane transporter protein is a LIdP lactate permease; a LctP membrane, a XylE xylose permease, or combinations thereof.

In an embodiment for better results, the DNA construct is obtained by ligating the DNA coding sequence for the prokaryotic membrane transporter protein between the N-terminal and the C-terminal DNA coding sequences of the eukaryotic membrane transporter protein.

embodiment for better results, the N-terminal coding DNA sequence is ligated before the initiation codon of the DNA coding sequence for the prokaryotic protein, and the C-terminal coding sequence is ligated after the penultimate codon of the DNA coding sequence for the prokaryotic protein

In an embodiment for better results, the method of the present disclosure further comprising the separation and/or purification of the prokaryotic membrane transporter protein.

Another aspect relates to a DNA construct comprising a DNA coding sequence for a prokaryotic membrane transporter protein is a permease, fused with the N-terminal or/and C-terminal DNA coding sequences of a eukaryotic membrane protein.

Another aspect relates to a eukaryotic host cell comprising the DNA construct of the present disclosure.

Another aspect relates to the use of the DNA construct or the eukaryotic host cell of the present disclosure as an increaser of cell transport capacity.

Another aspect relates to the use of the DNA construct or the eukaryotic host cell of the present disclosure as an increaser of the tolerance of eukaryotic organisms to intracellular compounds through the export of this molecule.

### BRIEF DESCRIPTION OF THE DRAWINGS

The following figures provide preferred embodiments for illustrating the description and should not be seen as limiting the scope of invention.

FIG. 1. Schematic representation of the DNA construction. The genetic construct is based in the genetic fusion of the N and/or C terminal coding DNA sequences of eukaryote membrane proteins with the DNA coding sequences of prokaryotic membrane transporter proteins at the beginning and the end of the protein DNA sequence. This DNA encodes the protein chimera required for the expression of prokaryotic membrane transporter proteins in eukaryotes.

FIG. 2. Subcellular localization of the constructs pNJ-Ildp-CJ-GFP and pNJ-Ictp-CJ-GFP evaluated by fluorescence microscopy. Cells were grown in YNB lactic acid 0.5% pH=5.5 at 30° C. to the middle-exponential phase and the GFP fluorescence was observed. Both lactate transporters are expressed and localized in the plasma membrane of S. cerevisiae W303-1A jen1Δady2Δ cells. The size of the scale bar is 7.5 μm.

FIG. 3. Growth tests of the yeast S. cerevisiae W303-1A jen1Δ ady2Δ cells expressing the plasmid pDS1, p416GPD, pNJ-LIdP-CJ-GFP, pLIdP, pNJ-LctP-CJ-GFP, pLctP. Cells grown in YNB Glucose 2% and YNB Lactic acid 0.5% at pH 5.5. Cells were diluted in sterilized water, the first drop corresponds to an optical density of 0.2 and the remaining dilutions are 1:10, 1:100 and 1:1000. The cells containing the plasmids p416GPD and pJen1-GFP are the negative and positive controls, respectively.

FIG. 4. Initial uptake rates of the radiolabelled 14C -Lactic acid at different concentrations. S. cerevisaie W303-1A jen1 Δady2Δ cells containing the plasmid pNJ-LctP-CJ-GFP and pNJ-LIdp-CJ-GFP were grown in YNB Lactic acid medium at pH 5.5 and 30° C., until mid-exponential growth phase. The pNJ-LctP-CJ-GFP has a Km of 0.17±0.03 mM and Vmax of 0.22±0.01 nmol s-1 mg-1 dry wt. Cells containing pNJ-LIdP-CJ-GFP, have a Km of 0.15±0.02 mM and Vmax of 0.2±0.01 nmol s-1 mg-1 dry wt. The positive control (pDS1-GFP) has a Km value of 0.27 and a Vmax of 0.23. The strains expressing the empty vector (p416GPD), pLIdP and pLctP displayed a Kd of 0.043±0.002 mM, 0.047±0.0025 mM and 0.047±0.0022 mM, respectively.

FIG. 5. Growth tests of the yeast S. cerevisiae EBY. 4000 cells expressing the plasmid, p416GPD, pNH-XylE-CH-GFP, pHxt1-GFP. Cells were grown in YNB Glucose 2% and Maltose 2% at 30° C. during 72 h. The cells containing the plasmids p416GPD and pHxt1-GFP are the negative and positive controls, respectively.

### DETAILED DESCRIPTION

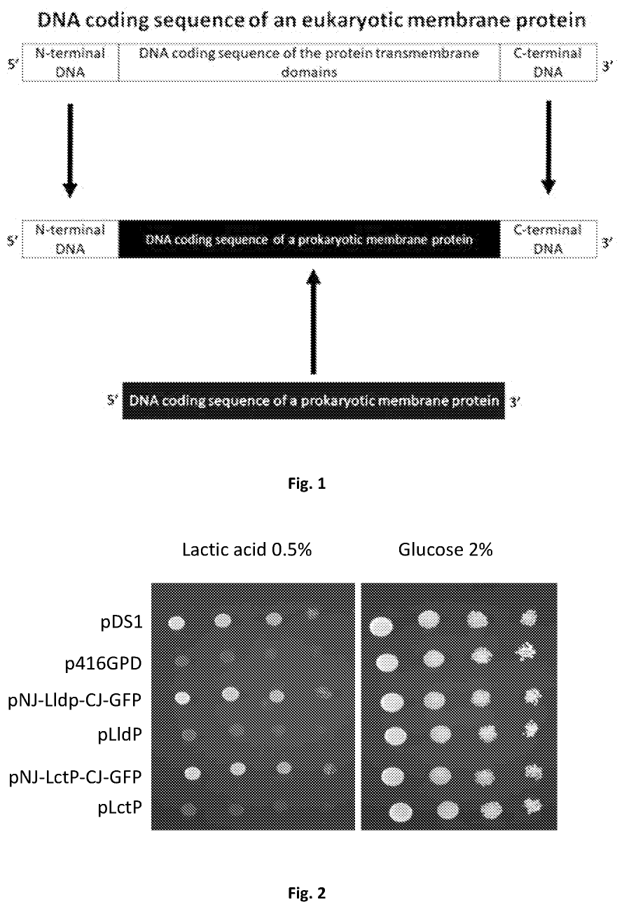

An aspect of the present disclosure is to create an expression system to functionally express prokaryotic membrane transporter proteins in eukaryote organisms. This expression system is based in the generation of a DNA construct that comprises the DNA sequence of a prokaryotic gene coding for a membrane protein fused with the DNA sequence coding for the N-terminal and/or C-terminal of a eukaryote membrane protein (FIG. 1). The N-terminal coding sequence is inserted before the prokaryotic protein initiation codon, and the C-terminal coding sequence right after the penultimate codon of the prokaryotic protein.

In this present disclosure, the N-terminal DNA coding sequences they are considering total or partial DNA sequences that range from the initial codon until the DNA sequence that codes for the first predicted transmembrane segment of a eukaryote membrane protein.

In this present disclosure, the C-terminal DNA coding sequences they are considering total or partial DNA sequences that range from the predicted last transmembrane segment of a eukaryote membrane protein until the last codon. Topological and secondary structure prediction should be performed to select the N-terminal and C-terminal DNA sequences from a eukaryotic membrane protein. The information collected through this in silico analysis will allow to infer on the number of transmembrane sequences, presence of protein domains and the length of the N and C termini. If information on membrane protein trafficking and regulation is available, it should also be considered in the process of N and C terminal DNA coding sequence selection. Ultimately, the information gathered will suggest the size of the N and C termini that will be fused with the prokaryotic membrane transporter protein DNA coding sequence. The N and C termini can belong to the same plasma membrane protein or to two different proteins, according to the properties of the original eukaryotic proteins and the desired applications.

In an embodiment, three prokaryotic transporters, LIdP, LctP and XylE, were selected and fused with the N and C termini of the S. cerevisiae transporters ScJen1 (LIdP and LctP) and Hxt1 (XylE) to generate the chimeras NJ-LIdP-CJ-GFP, NJ-LctP-CJ-GFP and NH-XylE-CH-GFP.

In order to experimentally validate the present invention, the inventors used two strains: the S. cerevisiae ady2Δ jen1Δ (Soares-Silva et al. 2007) and the S. cerevisiae EBY.4000

In an embodiment, the S. cerevisiae ady2Δ jen1Δ A strain under the conditions tested, is unable to actively transport and use efficiently carboxylic acids as sole carbon and energy source (Soares-Silva et al. 2007). This strain was used in the past to characterize several carboxylate transporters (Queiros, et al. 2007, Ribas D, et al. 2017, Soares-Silva, et al. 2015)

In an embodiment, the S. cerevisiae EBY.4000 strain is unable to growth in medium containing glucose as sole carbon and energy source (Wieczorke, et al. 1999).

In an embodiment, to confirm the successful heterologous expression of transporters in this system several studies were carried out as described in (Soares-Silva, et al. 2015): radiolabelled lactate uptake assays, growth assays in solid minimal medium with carboxylates or sugars as sole carbon source and fluorescence microscopy to detect the location of GFP fusion proteins.

### EXAMPLE I

Functional expression of the prokaryotic LIdP lactate permease in yeast by fusing the N-terminal and C-terminal of the ScJen1 lactate transporter to the LIdP transporter protein.

The present disclosure was firstly applied in the heterologous expression of the LIdP lactate transporter from E. coli in the eukaryotic host organism S. cerevisiae. As previously described the N- and C-terminals DNA coding sequences of ScJen1 were fused before the beginning and after the penultimate codon of the IldP gene, respectively (see sequences NJ-Ildp-CJ-GFP). The IldP gene was amplified by PCR from the E. coli genome with the Ld_1 and Ld_2 primers (Table 1) and then was insert in the pDS1-GFP vector linearized with Sphl (Soares-Silva, et al. 2007) by gap repair methodology, as described previously (Bessa, et al. 2012). This approach allows to generate a genetic construct composed sequentially by the ScJen1 N-terminal DNA coding sequence (from 1-423 nucleotides), the LIdP coding gene (from 1-1656 nucleotides) the ScJen1 C-terminal DNA coding sequence (from 1608-1848 nucleotides) and GFP coding gene (from 4-710 nucleotides), under the control of the GPD promoter (original vector p416GPD Mumberg 1995) which after translation will generate the NJ-LIdP-CJ-GFP protein. The resulting vector was transformed in the S. cerevisiae ady2Δjen1Δ strain. As a control the IldP gene was cloned in the p416GPD vector. For this construction the IldP gene was amplified from E. coli genomic DNA using the primers LIFWD and LIREV (Table 1) and inserted and ligated in the p416GPD vector using the restriction enzymes BamHI and Xbal.

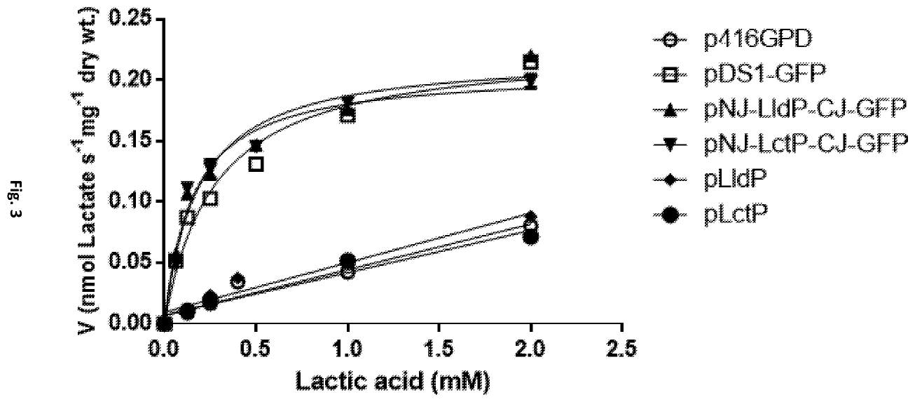

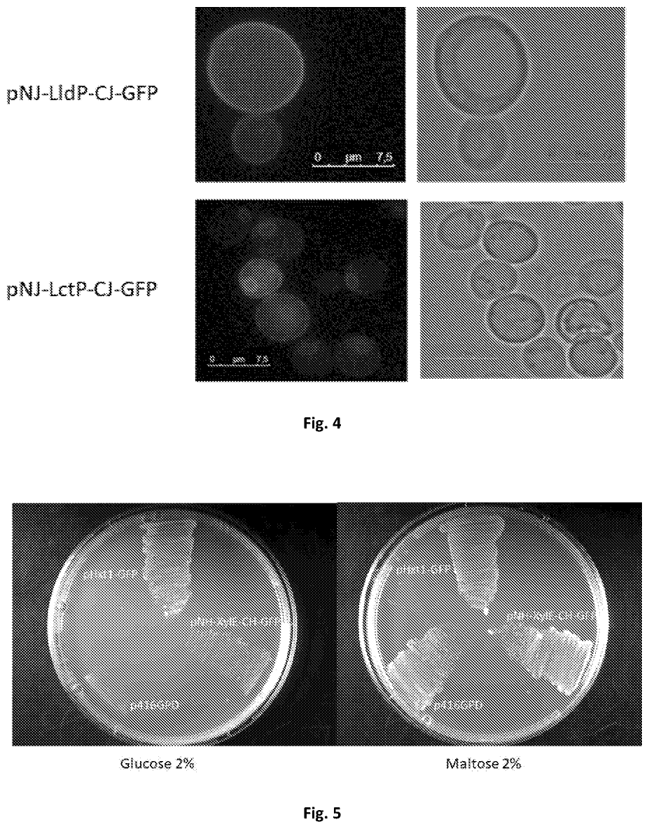

The growth of the S. cerevisiae ady2Δjen1Δ strain expressing the NJ-LldP-CJ-GFP protein and control strains were evaluated in YNB media (supplemented according to the required auxotrophies) containing lactic acid (0.5%) pH 5.5 at 18° C. The S. cerevisiae ady2Δjen1Δ strains expressing the native LIdP (pLIdP), the empty vector (p416GPD) and the ScJen1p (pDS1) were used as controls. The strain expressing NJ-LIdP-CJ-GFP was able to grow in minimal medium with lactic acid as sole carbon and energy source (FIG. 2) presenting a growth similar to the strain expressing ScJen1. The initial lactate uptake rates displayed by S. cerevisiae strains expressing pNJ-LIdP-CJ confirmed the data observed in growth tests (FIG. 3). Based on these results, kinetic parameters were determined for lactic acids uptake (pH 5.0). The expression of NJ-LIdP-CJ gene allowed the cells to transport labelled lactic acid by a mediated mechanism (Kₘ 0.15±0.02 mM; Vₘₐₓ0.2±0.01 nmol.s⁻¹.mg⁻¹.dry wt). The determined kinetic parameters were similar to the strain expressing ScJen1 (Kₘ0.27±0.04 mM; Vₘₐₓ0.23±0.01 nmol.s⁻¹.mg⁻¹.dry wt). The S. cerevisiae strain expressing the native Lldp presents a non-mediated transport mechanism for lactate, with a diffusion component equivalent to the strain expressing the empty vector (p416GPD), 0.043±0.002 mM and K_{d} 0.047±0.0025 mM, respectively. Fluorescence microscopy analysis of S. cerevisiae ady2Δ jen1Δ cells expressing NJ-LIdP-CJ protein tagged with GFP as a reporter gene revealed that the fusion protein was localized at the plasma membrane (FIG. 4).

### EXAMPLE II

Functional expression of the prokaryotic LctP membrane protein in yeast by adding the N-terminal and C-terminal of the ScJen1 lactate transporter.

A second example of the application of the present invention is the heterologous expression of the LctP putative lactate permease from S. aureus in the host eukaryotic organism S. cerevisiae. As described previously the N- and C-termini DNA coding sequences of ScJen1 were fused before the beginning and after the penultimate codon of the IctP gene, respectively. The IctP gene was amplified from E. coli genome with Lc_1 and Lc_2 primers (Table 1) and was inserted in the Sphl digested pJen1GFP vector (Soares-Silva, et al. 2007) by gap repair methodology, as described previously (Bessa, et al. 2012). As result a genetic construct was generated, which comprises sequentially the ScJen1 N-terminal DNA coding sequence (from 1-423 nucleotides), the LctP coding gene (from 1-1593 nucleotides) the ScJen1 C-terminal DNA coding sequence (from 1608-1848) and the GFP coding gene (from 4-710 nucleotides), which after translation generated the NJ-LctP-CJ-GFP protein. Then resulting vector pNJ-LctP-CJ-GFP was transformed in the yeast S. cerevisiae ady2Δjen1Δ strain.

As a control the IctP gene was cloned in the p416GPD vector. For this construct the IctP gene was amplified from S. aureus genomic DNA using the primers LcFWD and LcREV (Table 1) and inserted and ligated in the p416GPD vector using the restriction enzymes BamHI and EcoRI. Fluorescence microscopy analysis of S. cerevisiae ady2Δ jen1Δ NJ-LctP-CJ-GFP revealed that the chimeric protein was localized at the plasma membrane (FIG. 4). The growth of S. cerevisiae strains was tested in YNB media (supplemented according to the required auxotrophies) containing lactic acid 0.5% (pH 5.5). The S. cerevisiae ady2Δ jen1Δ NJ-LctP-CJ-GFP evidenced an improved growth compared to the control strains (FIG. 2). The initial lactate uptake rates displayed by cells expressing pNJ-LctP-CJ-GFP confirmed the data observed in the growth tests (FIG. 3). Based on these results, kinetic parameters were determined for lactic acids uptake (pH 5.0). The expression of NJ-LctP-CJ-GFP allowed the cells to transport labelled lactic acid by a mediated mechanism (Kₘ 0.17 ±0.03 mM; Vmax 0.22 ±0.01 nmol.s⁻¹.mg⁻¹.dry wt).

The S. cerevisiae strain expressing the native LcTp presents a non-mediated transport mechanism for lactate, with a diffusion component equivalent to the strain expressing the empty vector (p416GPD), 0.047±0.0022 mM and 0.043±0.002 mM respectively.

### EXAMPLE III

Functional expression of the prokaryotic XylE xylose permease in yeast by fusing the N-terminal and C-terminal of the Hxt1 glucose transporter to the XylE transporter protein.

A third example of the application of the present invention is the heterologous expression of the XylE xylose transporter from E. coli in the eukaryotic organism S. cerevisiae. In this experiment, the N- and C-terminals DNA coding sequences of Hxt1 were fused before the beginning and after the penultimate codon of the xa ligartylE gene, respectively (see sequence NJ-XylE-CJ-GFP). A synthetic codon optimized version for expression in S. cerevisiae of xylE gene (DNA sequences) was used in this work. The set of primers XylE1 and XylE2 primers (Table 1) were used to amplify the synthetic XylE. The resulting PCR product was inserted in the pHxt1-GFP vector linearized with BsaBI enzyme, by gap repair methodology, as described previously (Bessa, et al. 2012). This approach allows to generate a genetic construct composed sequentially by the Hxt1 N-terminal DNA coding sequence (from 1-177 nucleotides), the XylE coding gene (from 1-1473 nucleotides), the Hxt1 C-terminal DNA coding sequence (from 1539-1710 nucleotides), and the GFP coding gene (from 4-710 nucleotides), under the control of the GPD promoter (original vector p416GPD (Mumberg, Muller and Funk 1995)) which after translation will generate the NH-XylE-CH protein. The resulting vector was transformed in the S. cerevisiae EBY.4000 strain, which is unable to growth in medium containing glucose as sole carbon and energy source (Wieczorke, et al. 1999),It is noteworthy that S. cerevisiae is not able to growth in media containing xylose as sole carbon source. The pHxt1-GFP vector was used as a positive control. This construct was created by amplifying the HXT1 gene with HF and HR primers (Table 1) from S. cerevisiae genomic DNA. The PCR product was inserted and ligated in the p416GPD vector using the restriction enzymes BamHI and HindIII originating the pHxt1 vector. The GFP sequence was amplified with the primers Hxt1F and GFPR (Table 1) inserted in the pHxt1 vector linearized with HindIII enzyme, by gap repair methodology, as described previously.

The growth of the S. cerevisiae EBY.4000 strain expressing the NH-XylE-CJ-GFP protein and the control strains expressing XylE was evaluated in YNB media (supplemented according to the required auxotrophies) containing glucose (2%) at 30° C. (FIG. 6), which displayed a positive growth phenotype unlike the strain expressing the empty vector p416GPD (negative control), although with less biomass than the strain expressing the Hxt1 glucose transporter (positive control). This can result from a lower transport capacity of the xylE transporter for glucose, compared to the Hxt1.

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

Where singular forms of elements or features are used in the specification of the claims, the plural form is also included, and vice versa, if not specifically excluded. For example, the term “a sequence” or “the sequence” also includes the plural forms “sequences” or “the sequences,” and vice versa. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

TABLE 1
List of primers

| | Primer | |
|---|---|---|
| | name | Nucleotide sequence |
| Seq. ID 9 | Ld_1 | AACTGCGCAAAATGACATGGCAGAATTGGAACTATATGAATCTCTGGC |
| | | AACAA |
| Seq. ID 10 | Ld_2 | AATGTGAAGATGAAAACAGAACCAGTCAAGATAGCAGGAATCATCCAC |
| | | GT |
| Seq. ID 11 | Lc_1 | AACTGCGCAAAATGACATGGCAGAATTGGAACTATATGACACTACTTAC |
| | | TGTA |
| Seq. ID 12 | Lc_2 | AATGTGAAGATGAAAACAGAACCAGTCAAGATAGcGAATATTAACGTT |
| | | AGTA |
| Seq. ID 13 | XylE1 | CCCGCCGTTGCCCCTCCAAACACCGGAAAAATGAATACACAATACAACT |
| | | CTT |
| Seq. ID 14 | XylE2 | ACTTCTTCTAATGATAAACCTTTAGTTTCTGGCAGCGTAGCAGTTTG |
| Seq. ID 15 | L1FWD | GGGGGATCCATGAATCTCTGGCAACAA |
| Seq. ID 16 | L1REV | GGGGAATTCTTAAGGAATCATCCACGT |
| Seq. ID 17 | LcFWD | GGGGGATCCATGACACTACTTACTGTA |
| Seq. ID 18 | LcREV | GGGGAATTCTTAGAATATTAACGTTAGTA |
| Seq. ID 19 | HF | GATCCCCCGGGCTGCAGGAATTCGATATCAATGAATTCAACTCCCGATC |
| Seq. ID 20 | HR | CATGACTCGAGGTCGACGGTATCGATAAGCTTTATTTCCTGCTAAACAA |
| | | ACTC |
| Seq. ID 21 | Hxt1f | GACAACTCCAGTGAAAAGTTCTTCTCCTTTACTTTTCCTGCTAAACAA |
| Seq. ID 22 | GFPR | TTACATGACTCGAGGTCGACGGTATCGATAAGCTTGATATCGAACTATT |
| | | TGTATAGTTCATCCATGC |

DNA sequences

Seq. ID 1: JEN1 (S. cerevisiae)
>gi|330443667: 22234-24084 Saccharomyces cerevisiae S288c chromosome XI,
complete sequence
ATGTCGTCGTCAATTACAGATGAGAAAATATCTGGTGAACAGCAACAACCTGCTGGCAGAAA
ACTATACTATAACACAAGTACATTTGCAGAGCCTCCTCTAGTGGACGGAGAAGGTAACCCTAT
AAATTATGAGCCGGAAGTTTACAACCCGGATCACGAAAAGCTATACCATAACCCATCACTGCC
TGCACAATCAATTCAGGATACAAGAGATGATGAATTGCTGGAAAGAGTTTATAGCCAGGATC
AAGGTGTAGAGTATGAGGAAGATGAAGAGGATAAGCCAAACCTAAGCGCTGCGTCCATTAAA
AGTTATGCTTTAACGAGATTTACGTCCTTACTGCACATCCACGAGTTTTCTTGGGAGAATGTCA
ATCCCATACCCGAACTGCGCAAAATGACATGGCAGAATTGGAACTATTTTTTTATGGGTTATTT
TGCGTGGTTGTCTGCGGCTTGGGCCTTCTTTTGCGTTTCAGTATCAGTCGCTCCATTGGCTGAA
CTATATGACAGACCAACCAAGGACATCACCTGGGGGTTGGGATTGGTGTTATTTGTTCGTTCA
GCAGGTGCTGTCATATTTGGTTTATGGACAGATAAGTCTTCCAGAAAGTGGCCGTACATTACA
TGTTTGTTCTTATTTGTCATTGCACAACTCTGTACTCCATGGTGTGACACATACGAGAAATTTCT
GGGCGTAAGGTGGATAACCGGTATTGCTATGGGAGGAATTTACGGATGTGCTTCTGCAACAG
CGATTGAAGATGCACCTGTGAAAGCACGTTCGTTCCTATCAGGTCTATTTTTTTCTGCTTACGCT
ATGGGGTTCATATTTGCTATCATTTTTTACAGAGCCTTTGGCTACTTTAGGGATGATGGCTGGA
AAATATTGTTTTGGTTTAGTATTTTTCTACCAATTCTACTAATTTTCTGGAGATTGTTATGGCCT
GAAACGAAATACTTCACCAAGGTTTTGAAAGCCCGTAAATTAATATTGAGTGACGCAGTGAAA
GCTAATGGTGGCGAGCCTCTACCAAAAGCCAACTTTAAACAAAAGATGGTATCCATGAAGAG
AACAGTTCAAAAGTACTGGTTGTTGTTCGCATATTTGGTTGTTTTATTGGTGGGTCCAAATTAC
TTGACTCATGCTTCTCAAGACTTGTTGCCAACCATGCTGCGTGCCCAATTAGGCCTATCCAAGG
ATGCTGTCACTGTCATTGTAGTGGTTACCAACATCGGTGCTATTTGTGGGGGTATGATATTTGG
ACAGTTCATGGAAGTTACTGGAAGAAGATTAGGCCTATTGATTGCATGCACAATGGGTGGTT
GCTTCACCTACCCTGCATTTATGTTGAGAAGCGAAAAGGCTATATTAGGTGCCGGTTTCATGTT
ATATTTTTGTGTCTTTGGTGTCTGGGGTATCCTGCCCATTCACCTTGCAGAGTTGGCCCCTGCT
GATGCAAGGGCTTTGGTTGCCGGTTTATCTTACCAGCTAGGTAATCTAGCTTCTGCAGCGGCTT
CCACGATTGAGACACAGTTAGCTGATAGATACCCATTAGAAAGAGATGCCTCTGGTGCTGTGA
TTAAAGAAGATTATGCCAAAGTTATGGCTATCTTGACTGGTTCTGTTTTCATCTTCACATTTGCT
TGTGTTTTTGTTGGCCATGAGAAATTCCATCGTGATTTGTCCTCTCCTGTTATGAAGAAATATAT
AAACCAAGTGGAAGAATACGAAGCCGATGGTCTTTCGATTAGTGACATTGTTGAACAAAAGA
CGGAATGTGCTTCAGTGAAGATGATTGATTCGAACGTCTCAAAGACATATGAGGAGCATATTG
AGACCGTTTAA
Seq. ID 2: HXT1 (S. cerevisiae)
>NC_001140.6: c292625-290913 Saccharomyces cerevisiae S288c chromosome VIII,
complete sequence
ATGAATTCAACTCCCGATCTAATATCTCCTCAGAAATCCAATTCATCCAACTCATATGAATTGGA
ATCTGGTCGTTCAAAGGCCATGAATACTCCAGAAGGTAAAAATGAAAGTTTTCACGACAACTT
AAGTGAAAGTCAAGTGCAACCCGCCGTTGCCCCTCCAAACACCGGAAAAGGTGTCTACGTAAC
GGTTTCTATCTGTTGTGTTATGGTTGCTTTCGGTGGTTTCATATTTGGATGGGATACTGGTACC
ATTTCTGGTTTTGTTGCTCAAACTGATTTTCTAAGAAGATTTGGTATGAAGCACCACGACGGTA
GTCATTACTTGTCCAAGGTGAGAACTGGTTTAATTGTCTCTATTTTTAACATTGGTTGTGCCATT
GGTGGTATCGTCTTAGCCAAGCTAGGTGATATGTATGGTCGTAGAATCGGTTTGATTGTCGTT
GTAGTAATCTACACTATCGGTATCATTATTCAAATAGCCTCGATCAACAAGTGGTACCAATATT
TCATTGGTAGAATTATCTCTGGTTTAGGTGTCGGTGGTATCACAGTTTTATCTCCCATGCTAAT
ATCTGAGGTCGCCCCCAGTGAAATGAGAGGCACCTTGGTTTCATGTTACCAAGTCATGATTAC
TTTAGGTATTTTCTTAGGTTACTGTACCAATTTTGGTACCAAGAATTACTCAAACTCTGTCCAAT
GGAGAGTTCCATTAGGTTTGTGTTTCGCCTGGGCCTTATTTATGATTGGTGGTATGATGTTTGT
TCCTGAATCTCCACGTTATTTGGTTGAAGCTGGCAGAATCGACGAAGCCAGGGCTTCTTTAGC
TAAAGTTAACAAATGCCCACCTGACCATCCATACATTCAATATGAGTTGGAAACTATCGAAGCC
AGTGTCGAAGAAATGA
GAGCCGCTGGTACTGCATCTTGGGGCGAATTATTCACTGGTAAACCAGCCATGTTTCAACGTA
CTATGATGGGTATCATGATTCAATCTCTACAACAATTAACTGGTGATAACTATTTCTTCTACTAC
GGTACCATTGTTTTCCAGGCTGTCGGTTTAAGTGACTCTTTTGAAACTTCTATTGTCTTTGGTGT
CGTCAACTTCTTCTCCACTTGTTGTTCTCTGTACACCGTTGACCGTTTTGGCCGTCGTAACTGTT
TGATGTGGGGTGCTGTCGGTATGGTCTGCTGTTATGTTGTCTATGCCTCTGTTGGTGTTACCAG
ATTATGGCCAAACGGTCAAGATCAACCATCTTCAAAGGGTGCTGGTAACTGTATGATTGTTTTC
GCATGTTTCTACATTTTCTGTTTCGCTACTACCTGGGCCCCAATTGCTTACGTTGTTATTTCAGA
ATGTTTCCCATTAAGAGTCAAATCCAAGTGTATGTCTATTGCCAGTGCTGCTAACTGGATCTGG
GGTTTCTTGATTAGTTTCTTCACCCCATTTATTACTGGTGCCATCAACTTCTACTACGGTTACGTT
TTCATGGGCTGTATGGTTTTCGCTTACTTTTACGTCTTTTTCTTCGTTCCAGAAACTAAAGGTTT
ATCATTAGAAGAAGTTAATGATATGTACGCCGAAGGTGTTCTACCATGGAAATCAGCTTCCTG
GGTTCCAGTATCCAAGAGAGGCGCTGACTACAACGCTGATGACCTAATGCATGATGACCAACC
ATTTTACAAGAGTTTGTTTAGCAGGAAATAA
Seq. ID 3: IldP (E. coli)
>gi|556503834: 3777399-3779054 Escherichia coli str. K-12 substr. MG1655, complete
genome
ATGAATCTCTGGCAACAAAACTACGATCCCGCCGGGAATATCTGGCTTTCCAGTCTGATAGCA
TCGCTTCCCATCCTGTTTTTCTTCTTTGCGCTGATTAAGCTCAAACTGAAAGGATACGTCGCCGC
CTCGTGGACGGTGGCAATCGCCCTTGCCGTGGCTTTGCTGTTCTATAAAATGCCGGTCGCTAA
CGCGCTGGCCTCGGTGGTTTATGGTTTCTTCTACGGGTTGTGGCCCATCGCGTGGATCATTATT
GCAGCGGTGTTCGTCTATAAGATCTCGGTGAAAACCGGGCAGTTTGACATCATTCGCTCGTCT
ATTCTTTCGATAACCCCTGACCAGCGTCTGCAAATGCTGATCGTCGGTTTCTGTTTCGGCGCGT
TCCTTGAAGGAGCCGCAGGCTTTGGCGCACCGGTAGCAATTACCGCCGCATTGCTGGTCGGCC
TGGGTTTTAAACCGCTGTACGCCGCCGGGCTGTGCCTGATTGTTAACACCGCGCCAGTGGCAT
TTGGTGCGATGGGCATTCCAATCCTGGTTGCCGGACAGGTAACAGGTATCGACAGCTTTGAG
ATTGGTCAGATGGTGGGGCGGCAGCTACCGTTTATGACCATTATCGTGCTGTTCTGGATCATG
GCGATTATGGACGGCTGGCGCGGTATCAAAGAGACGTGGCCTGCGGTCGTGGTTGCGGGCG
GCTCGTTTGCCATCGCTCAGTACCTTAGCTCTAACTTCATTGGGCCGGAGCTGCCGGACATTAT
CTCTTCGCTGGTATCACTGCTCTGCCTGACGCTGTTCCTCAAACGCTGGCAGCCAGTGCGTGTA
TTCCGTTTTGGTGATTTGGGGGCGTCACAGGTTGATATGACGCTGGCCCACACCGGTTACACT
GCGGGTCAGGTGTTACGTGCCTGGACACCGTTCCTGTTCCTGACAGCTACCGTAACACTGTGG
AGTATCCCGCCGTTTAAAGCCCTGTTCGCATCGGGTGGCGCGCTGTATGAGTGGGTGATCAAT
ATTCCGGTGCCGTACCTCGATAAACTGGTTGCCCGTATGCCGCCAGTGGTCAGCGAGGCTACA
GCCTATGCCGCCGTGTTTAAGTTTGACTGGTTCTCTGCCACCGGCACCGCCATTCTGTTTGCTG
CACTGCTCTCGATTGTCTGGCTGAAGATGAAACCGTCTGACGCTATCAGCACCTTCGGCAGCA
CGCTGAAAGAACTGGCTCTGCCCATCTACTCCATCGGTATGGTGCTGGCATTCGCCTTTATTTC
GAACTATTCCGGACTGTCATCAACACTGGCGCTGGCACTGGCGCACACCGGTCATGCATTCAC
CTTCTTCTCGCCGTTCCTCGGCTGGCTGGGGGTATTCCTGACCGGGTCGGATACCTCATCTAAC
GCCCTGTTCGCCGCGCTGCAAGCCACCGCAGCACAACAAATTGGCGTCTCTGATCTGTTGCTG
GTTGCCGCCAATACCACCGGTGGCGTCACCGGTAAGATGATCTCCCCGCAATCTATCGCTATC
GCCTGTGCGGCGGTAGGCCTGGTGGGCAAAGAGTCTGATTTGTTCCGCTTTACTGTCAAACAC
AGCCTGATCTTCACCTGTATAGTGGGCGTGATCACCACGCTTCAGGCTTATGTCTTAACGTGGA
TGATTCCTTAA
Seq. ID 4: Ici-P (S. aureus)
>ENA|ABD29252|ABD29252.1 Staphylococcus aureus subsp. aureus NCTC 8325 L-
lactate permease
ATGACACTACTTACTGTAAATCCATTCGATAATGTCGGATTATCAGCCTTAGTTGCAGCAGTAC
CTATTATTTTATTTTTATTATGCTTAACCGTTTTTAAAATGAAAGGCATTTATGCAGCATTGACA
ACTTTGGTTGTTACATTGATTGTGGCTTTATTTGTATTTGAATTACCAGCGCGTGTATCAGCAG
GTGCGATTACAGAAGGCGTTGTTGCCGGTATTTTCCCAATAGGATATATCGTTTTAATGGCAG
TTTGGTTATATAAAGTTTCTATTAAAACAGGACAATTTTCTATTATTCAAGATAGTATTGCAAGT
ATTTCAGTGGACCAAAGAATCCAACTATTATTAATTGGATTTTGTTTCAACGCATTTTTAGAAG
GTGCAGCAGGATTTGGTGTGCCAATTGCGATTTGTGCAGTATTATTAATTCAACTTGGATTTGA
ACCATTAAAAGCAGCGATGTTATGTTTAATTGCTAATGGTGCGGCGGGTGCCTTTGGTGCAAT
TGGTTTACCAGTTAGTATTATTGATACGTTTAACTTAAGTGGAGGCGTTACAACATTAGATGTT
GCGAGATACTCAGCATTAACACTTCCAATTTTAAACTTTATTATTCCATTTGTTTTAGTATTCATT
GTAGATGGTATGAAAGGTATTAAAGAAATTTTACCTGTCATTTTAACAGTGAGTGGTACATAT
ACTGGATTACAATTATTATTAACAATATTCCATGGTCCAGAACTAGCAGACATTATTCCATCACT
AGCAACAATGGTGGTGTTAGCATTTGTTTGTCGTAAATTTAAACCGAAAAACATTTTCAGATTG
GAAGCGTCTGAACATAAAATTCAAAAACGAACGCCTAAAGAAATTGTCTTTGCTTGGAGTCCG
TTCGTAATTTTAACTGCCTTTGTATTAGTATGGAGTGCACCATTCTTCAAAAAATTATTCCAACC
TGGAGGTGCACTTGAAAGTTTAGTAATAAAATTGCCAATTCCAAATACTGTGAGTGATTTATC
GCCTAAAGGAATTGCGTTGCGTCTCGATTTAATTGGTGCAACTGGGACAGCGATTTTATTAAC
AGTAATTATTACAATTTTAATTACGAAGTTAAAATGGAAAAGTGCAGGTGCTTTATTGGTCGA
AGCAATTAAAGAATTATGGTTACCGATCCTTACAATTTCAGCTATCCTAGCTATTGCTAAAGTT
ATGACATACGGTGGTTTGACTGTAGCAATTGGACAAGGTATTGCTAAAGCGGGAGCAATTTTC
CCATTATTCTCTCCAGTATTAGGTTGGATTGGTGTGTTTATGACTGGTTCAGTTGTAAATAACA
ATACTTTATTCGCACCTATTCAAGCGACAGTGGCACAACAAATTTCAACAAGCGGTTCATTACT
TGTGGCAGCTAACACTGCAGGTGGTGTAGCAGCGAAACTTATTTCACCACAATCAATTGCCAT
TGCGACTGCAGCTGTTAAAAAAGTTGGTGAAGAATCTGCATTATTAAAAATGACGTTAAAATA
CAGTATTATATTTGTTGCTTTTATTTGTGTTTGGACGTTTATACTAACGTTAATATTCTAA
Seq. ID 5: Synthetic xylE
ATGAATACACAATACAACTCTTCATACATTTTCTCTATCACTTTGGTTGCTACATTAGGTGGTTT
GTTGTTCGGTTACGATACTGCAGTTATTTCTGGTACAGTTGAATCATTGAACACTGTTTTCGTT
GCTCCACAAAATTTGTCTGAATCAGCTGCAAATTCTTTGTTAGGTTTTTGTGTTGCTTCAGCATT
GATTGGTTGTATTATTGGTGGTGCATTAGGTGGTTACTGTTCTAACAGATTCGGTAGAAGAGA
TTCATTGAAGATCGCTGCAGTTTTGTTTTTCATCTCTGGTGTTGGTTCAGCTTGGCCAGAATTG
GGTTTTACATCTATTAATCCAGATAACACTGTTCCAGTTTATTTGGCAGGTTACGTTCCAGAATT
CGTTATCTATAGAATCATCGGTGGTATTGGTGTTGGTTTGGCTTCTATGTTATCACCAATGTAC
ATTGCAGAATTGGCTCCAGCACATATTCGTGGTAAATTGGTTTCTTTTAATCAATTCGCTATCAT
CTTCGGTCAATTGTTAGTTTATTGTGTTAATTACTTTATTGCTAGATCTGGTGACGCATCATGGT
TGAATACTGACGGCTGGCGTTATATGTTTGCCTCGGAATGTATCCCTGCACTGCTGTTCTTAAT
GCTGCTGTATACCGTGCCAGAAAGTCCTCGCTGGCTGATGTCGCGCGGCAAGCAAGAACAGG
CGGAAGGTATCCTGCGCAAAATTATGGGCAACACGCTTGCAACTCAGGCAGTACAGGAAATT
AAACACTCCCTGGATCATGGCCGCAAAACCGGTGGTCGTCTGCTGATGTTTGGCGTGGGCGT
GATTGTAATCGGCGTAATGCTCTCCATCTTCCAGCAATTTGTCGGCATCAATGTGGTGCTGTAC
TACGCGCCGGAAGTGTTCAAAACGCTGGGGGCCAGCACGGATATCGCGCTGTTGCAGACCAT
TATTGTCGGAGTTATCAACCTCACCTTCACCGTTCTGGCAATTATGACGGTGGATAAATTTGGT
CGTAAGCCACTGCAAATTATCGGCGCACTCGGAATGGCAATCGGTATGTTTAGCCTCGGTACC
GCGTTTTACACTCAGGCACCGGGTATTGTGGCGCTACTGTCGATGCTGTTCTATGTTGCCGCCT
TTGCCATGTCCTGGGGTCCGGTATGCTGGGTACTGCTGTCGGAAATCTTCCCGAATGCTATTC
GTGGTAAAGCGCTGGCAATCGCGGTGGCGGCCCAGTGGCTGGCGAACTACTTCGTCTCCTGG
ACCTTCCCGATGATGGACAAAAACTCCTGGCTGGTGGCCCATTTCCACAACGGTTTCTCCTACT
GGATTTACGGTTGTATGGGCGTTCTGGCAGCACTGTTTATGTGGAAATTTGTCCCGGAAACCA
AAGGTAAAACCCTTGAGGAGCTGGAAGCGCTCTGGGAACCGGAAACGAAGAAAACACAACA
AACTGCTACGCTG
Seq. ID 6: Coding sequence NJ-LldP-CJ-GFP
ATGTCGTCGTCAATTACAGATGAGAAAATATCTGGTGAACAGCAACAACCTGCTGGCAGAAA
ACTATACTATAACACAAGTACATTTGCAGAGCCTCCTCTAGTGGACGAAGAAGGTAACCCTAT
AAATTATGAGCCGGAAGTTTACAACCCGGATCACGAAAAGCTATACCATAACCCATCACTGCC
TGCACAATCAATTCAGGATACAAGAGATGATGAATTGCTGGAAAGAGTTTATAGCCAGGATC
AAGGTGTAGAGTATGAGGAAGATGAAGAGGATAAGCCAAACCTAAGCGCTGCGTCCATTAAA
AGTTATGCTTTAACGAGATTTACGTCCTTACTGCACATCCACGAGTTTTCTTGGGAGAATGTCA
ATCCCATACCCGAACTGCGCAAAATGACATGGCAGAATTGGAACTATATGAATCTCTGGCAAC
AAAACTACGATCCCGCCGGGAATATCTGGCTTTCCAGTCTGATAGCATCGCTTCCCATCCTGTT
TTTCTTCTTTGCGCTGATTAAGCTCAAACTGAAAGGATACGTCGCCGCCTCGTGGACGGTGGC
AATCGCCCTTGCCGTGGCTTTGCTGTTCTATAAAATGCCGGTCGCTAACGCGCTGGCCTCGGT
GGTTTATGGTTTCTTCTACGGGTTGTGGCCCATCGCGTGGATCATTATTGCAGCGGTGTTCGTC
TATAAGATCTCGGTGAAAACCGGGCAGTTTGACATCATTCGCTCGTCTATTCTTTCGATAACCC
CTGACCAGCGTCTGCAAATGCTGATCGTCGGTTTCTGTTTCGGCGCGTTCCTTGAAGGAGCCG
CAGGCTTTGGCGCACCGGTAGCAATTACCGCCGCATTGCTGGTCGGCCTGGGTTTTAAACCGC
TGTACGCCGCCGGGCTGTGCCTGATTGTTAACACCGCGCCAGTGGCATTTGGTGCGATGGGC
ATTCCAATCCTGGTTGCCGGACAGGTAACAGGTATCGACAGCTTTGAGATTGGTCAGATGGTG
GGGCGGCAGCTACCGTTTATGACCATTATCGTGCTGTTCTGGATCATGGCGATTATGGACGGC
TGGCGCGGTATCAAAGAGACGTGGCCTGCGGTCGTGGTTGCGGGCGGCTCGTTTGCCATCGC
TCAGTACCTTAGCTCTAACTTCATTGGGCCGGAGCTGCCGGACATTATCTCTTCGCTGGTATCA
CTGCTCTGCCTGACGCTGTTCCTCAAACGCTGGCAGCCAGTGCGTGTATTCCGT1TGGTGAT
TGGGGGCGTCACAGGTTGATATGACGCTGGCCCACACCGGTTACACTGCGGGTCAGGTGTTA
CGTGCCTGGACACCGTTCCTGTTCCTGACAGCTACCGTAACACTGTGGAGTATCCCGCCGTTTA
AAGCCCTGTTCGCATCGGGTGGCGCGCTGTATGAGTGGGTGATCAATATTCCGGTGCCGTACC
TCGATAAACTGGTTGCCCGTATGCCGCCAGTGGTCAGCGAGGCTACAGCCTATGCCGCCGTGT
TTAAGTTTGACTGGTTCTCTGCCACCGGCACCGCCATTCTGTTTGCTGCACTGCTCTCGATTGTC
TGGCTGAAGATGAAACCGTCTGACGCTATCAGCACCTTCGGCAGCACGCTGAAAGAACTGGC
TCTGCCCATCTACTCCATCGGTATGGTGCTGGCATTCGCCTTTATTTCGAACTATTCCGGACTGT
CATCAACACTGGCGCTGGCACTGGCGCACACCGGTCATGCATTCACCTTCTTCTCGCCGTTCCT
CGGCTGGCTGGGGGTATTCCTGACCGGGTCGGATACCTCATCTAACGCCCTGTTCGCCGCGCT
GCAAGCCACCGCAGCACAACAAATTGGCGTCTCTGATCTGTTGCTGGTTGCCGCCAATACCAC
CGGTGGCGTCACCGGTAAGATGATCTCCCCGCAATCTATCGCTATCGCCTGTGCGGCGGTAGG
CCTGGTGGGCAAAGAGTCTGATTTGTTCCGCTTTACTGTCAAACACAGCCTGATCTTCACCTGT
ATAGTGGGCGTGATCACCACGCTTCAGGCTTATGTCTTAACGTGGATGATTCCTATGGCTATCT
TGACTGGTTCTGTTTTCATCTTCACATTTGCTTGTGTTTTTGTTGGCCATGAGAAATTCCATCGT
GATTTGTCCTCTCCTGTTATGAAGAAATATATAAACCAAGTGGAAGAATACGAAGCCGATGGT
CTTTCGATTAGTGACATTGTTGAACAAAAGACGGAATGTGCTTCAGTGAAGATGATTGATTCG
AACGTCTCAAAGACATATGAGGAGCATATTGAGACCGTTAGTAAAGGAGAAGAACTTTTCACT
GGAGTTGTCCCAATTCTTGTTGAATTAGATGGTGATGTTAATGGGCACAAATTTTCTGTCAGTG
GAGAGGGTGAAGGTGATGCAACATACGGAAAACTTACCCTTAAATTTATTTGCACTACTGGAA
AACTACCTGTTCCATGGCCAACACTTGTCACTACTTTCACTTATGGTGTTCAATGCTTTTCAAGA
TACCCAGATCATATGAAACGGCATGACTTTTTCAAGAGTGCCATGCCCGAAGGTTATGTACAG
GAAAGAACTATATTTTTCAAAGATGACGGGAACTACAAGACACGTGCTGAAGTCAAGTTTGAA
GGTGATACCCTTGTTAATAGAATCGAGTTAAAAGGTATTGATTTTAAAGAAGATGGAAACATT
CTTGGACACAAATTGGAATACAACTATAACTCACACAATGTATACATCATGGCAGACAAACAA
AAGAATGGAATCAAAGTTAACTTCAAAATTAGACACAACATTGAAGATGGAAGCGTTCAACTA
GCAGACCATTATCAACAAAATACTCCAATTGGCGATGGCCCTGTCCTTTTACCAGACAACCATT
ACCTGTCCACACAATCTGCCCTTTCGAAAGATCCCAACGAAAAGAGAGACCACATGGTCCTTCT
TGAGTTTGTAACAGCTGCTGGGATTACACATGGCATGGATGAACTATACAAATAG
Seq. ID 7: Coding sequence NJ-LctP-CJ-GFP
ATGTCGTCGTCAATTACAGATGAGAAAATATCTGGTGAACAGCAACAACCTGCTGGCAGAAA
ACTATACTATAACACAAGTACATTTGCAGAGCCTCCTCTAGTGGACGAAGAAGGTAACCCTAT
AAATTATGAGCCGGAAGTTTACAACCCGGATCACGAAAAGCTATACCATAACCCATCACTGCC
TGCACAATCAATTCAGGATACAAGAGATGATGAATTGCTGGAAAGAGTTTATAGCCAGGATC
AAGGTGTAGAGTATGAGGAAGATGAAGAGGATAAGCCAAACCTAAGCGCTGCGTCCATTAAA
AGTTATGCTTTAACGAGATTTACGTCCTTACTGCACATCCACGAGTTTTCTTGGGAGAATGTCA
ATCCCATACCCGAACTGCGCAAAATGACATGGCAGAATTGGAACTATATGACACTACTTACTG
TAAATCCATTCGATAATGTCGGATTATCAGCCTTAGTTGCAGCAGTACCTATTATTTTATTTTTA
TTATGCTTAACCGTTTTTAAAATGAAAGGCATTTATGCAGCATTGACAACTTTGGTTGTTACATT
GATTGTGGCTTTATTTGTATTTGAATTACCAGCGCGTGTATCAGCAGGTGCGATTACAGAAGG
CGTTGTTGCCGGTATTTTCCCAATAGGATATATCGTTTTAATGGCAGTTTGGTTATATAAAGTTT
CTATTAAAACAGGACAATTTTCTATTATTCAAGATAGTATTGCAAGTATTTCAGTGGACCAAAG
AATCCAACTATTATTAATTGGATTTTGTTTCAACGCATTTTTAGAAGGTGCAGCAGGATTTGGT
GTGCCAATTGCGATTTGTGCAGTATTATTAATTCAACTTGGATTTGAACCATTAAAAGCAGCGA
TGTTATGTTTAATTGCTAATGGTGCGGCGGGTGCCTTTGGTGCAATTGGTTTACCAGTTAGTAT
TATTGATACGTTTAACTTAAGTGGAGGCGTTACAACATTAGATGTTGCGAGATACTCAGCATT
AACACTTCCAATTTTAAACTTTATTATTCCATTTGTTTTAGTATTCATTGTAGATGGTATGAAAG
GTATTAAAGAAATTTTACCTGTCATTTTAACAGTGAGTGGTACATATACTGGATTACAATTATT
ATTAACAATATTCCATGGTCCAGAACTAGCAGACATTATTCCATCACTAGCAACAATGGTGGTG
TTAGCATTTGTTTGTCGTAAATTTAAACCGAAAAACATTTTCAGATTGGAAGCGTCTGAACATA
AAATTCAAAAACGAACGCCTAAAGAAATTGTCTTTGCTTGGAGTCCGTTCGTAATTTTAACTGC
CTTTGTATTAGTATGGAGTGCACCATTCTTCAAAAAATTATTCCAACCTGGAGGTGCACTTGAA
AGTTTAGTAATAAAATTGCCAATTCCAAATACTGTGAGTGATTTATCGCCTAAAGGAATTGCGT
TGCGTCTCGATTTAATTGGTGCAACTGGGACAGCGATTTTATTAACAGTAATTATTACAATTTT
AATTACGAAGTTAAAATGGAAAAGTGCAGGTGCTTTATTGGTCGAAGCAATTAAAGAATTATG
GTTACCGATCCTTACAATTTCAGCTATCCTAGCTATTGCTAAAGTTATGACATACGGTGGTTTG
ACTGTAGCAATTGGACAAGGTATTGCTAAAGCGGGAGCAATTTTCCCATTATTCTCTCCAGTAT
TAGGTTGGATTGGTGTGTTTATGACTGGTTCAGTTGTAAATAACAATACTTTATTCGCACCTAT
TCAAGCGACAGTGGCACAACAAATTTCAACAAGCGGTTCATTACTTGTGGCAGCTAACACTGC
AGGTGGTGTAGCAGCGAAACTTATTTCACCACAATCAATTGCCATTGCGACTGCAGCTGTTAA
AAAAGTTGGTGAAGAATCTGCATTATTAAAAATGACGTTAAAATACAGTATTATATTTGTTGCT
TTTATTTGTGTTTGGACGTTTATACTAACGTTAATATTCTATGGCTATCTTGACTGGTTCTGTTTT
CATCTTCACATTTGCTTGTGTTTTTGTTGGCCATGAGAAATTCCATCGTGATTTGTCCTCTCCTG
TTATGAAGAAATATATAAACCAAGTGGAAGAATACGAAGCCGATGGTCTTTCGATTAGTGACA
TTGTTGAACAAAAGACGGAATGTGCTTCAGTGAAGATGATTGATTCGAACGTCTCAAAGACAT
ATGAGGAGCATATTGAGACCGTTAGTAAAGGAGAAGAACTTTTCACTGGAGTTGTCCCAATTC
TTGTTGAATTAGATGGTGATGTTAATGGGCACAAATTTTCTGTCAGTGGAGAGGGTGAAGGT
GATGCAACATACGGAAAACTTACCCTTAAATTTATTTGCACTACTGGAAAACTACCTGTTCCAT
GGCCAACACTTGTCACTACTTTCACTTATGGTGTTCAATGCTTTTCAAGATACCCAGATCATATG
AAACGGCATGACTTTTTCAAGAGTGCCATGCCCGAAGGTTATGTACAGGAAAGAACTATATTT
TTCAAAGATGACGGGAACTACAAGACACGTGCTGAAGTCAAGTTTGAAGGTGATACCCTTGTT
AATAGAATCGAGTTAAAAGGTATTGATTTTAAAGAAGATGGAAACATTCTTGGACACAAATTG
GAATACAACTATAACTCACACAATGTATACATCATGGCAGACAAACAAAAGAATGGAATCAAA
GTTAACTTCAAAATTAGACACAACATTGAAGATGGAAGCGTTCAACTAGCAGACCATTATCAA
CAAAATACTCCAATTGGCGATGGCCCTGTCCTTTTACCAGACAACCATTACCTGTCCACACAAT
CTGCCCTTTCGAAAGATCCCAACGAAAAGAGAGACCACATGGTCCTTCTTGAGTTTGTAACAG
CTGCTGGGATTACACATGGCATGGATGAACTATACAAATAG
Seq. ID 8: Coding sequence NH-XylE-CH
ATGAATTCAACTCCCGATCTAATATCTCCTCAGAAATCCAATTCATCCAACTCATATGAATTGGA
ATCTGGTCGTTCAAAGGCCATGAATACTCCAGAAGGTAAAAATGAAAGTTTTCACGACAACTT
AAGTGAAAGTCAAGTGCAACCCGCCGTTGCCCCTCCAAACACCGGAAAAATGAATACACAATA
CAACTCTTCATACATTTTCTCTATCACTTTGGTTGCTACATTAGGTGGTTTGTTGTTCGGTTACG
ATACTGCAGTTATTTCTGGTACAGTTGAATCATTGAACACTGTTTTCGTTGCTCCACAAAATTTG
TCTGAATCAGCTGCAAATTCTTTGTTAGGTTTTTGTGTTGCTTCAGCATTGATTGGTTGTATTAT
TGGTGGTGCATTAGGTGGTTACTGTTCTAACAGATTCGGTAGAAGAGATTCATTGAAGATCGC
TGCAGTTTTGTTTTTCATCTCTGGTGTTGGTTCAGCTTGGCCAGAATTGGGTTTTACATCTATTA
ATCCAGATAACACTGTTCCAGTTTATTTGGCAGGTTACGTTCCAGAATTCGTTATCTATAGAAT
CATCGGTGGTATTGGTGTTGGTTTGGCTTCTATGTTATCACCAATGTACATTGCAGAATTGGCT
CCAGCACATATTCGTGGTAAATTGGTTTCTTTTAATCAATTCGCTATCATCTTCGGTCAATTGTT
AGTTTATTGTGTTAATTACTTTATTGCTAGATCTGGTGACGCATCATGGTTGAATACTGACGGC
TGGCGTTATATGTTTGCCTCGGAATGTATCCCTGCACTGCTGTTCTTAATGCTGCTGTATACCGT
GCCAGAAAGTCCTCGCTGGCTGATGTCGCGCGGCAAGCAAGAACAGGCGGAAGGTATCCTGC
GCAAAATTATGGGCAACACGCTTGCAACTCAGGCAGTACAGGAAATTAAACACTCCCTGGATC
ATGGCCGCAAAACCGGTGGTCGTCTGCTGATGTTTGGCGTGGGCGTGATTGTAATCGGCGTA
ATGCTCTCCATCTTCCAGCAATTTGTCGGCATCAATGTGGTGCTGTACTACGCGCCGGAAGTGT
TCAAAACGCTGGGGGCCAGCACGGATATCGCGCTGTTGCAGACCATTATTGTCGGAGTTATCA
ACCTCACCTTCACCGTTCTGGCAATTATGACGGTGGATAAATTTGGTCGTAAGCCACTGCAAAT
TATCGGCGCACTCGGAATGGCAATCGGTATGTTTAGCCTCGGTACCGCGTTTTACACTCAGGC
ACCGGGTATTGTGGCGCTACTGTCGATGCTGTTCTATGTTGCCGCCTTTGCCATGTCCTGGGGT
CCGGTATGCTGGGTACTGCTGTCGGAAATCTTCCCGAATGCTATTCGTGGTAAAGCGCTGGCA
ATCGCGGTGGCGGCCCAGTGGCTGGCGAACTACTTCGTCTCCTGGACCTTCCCGATGATGGAC
AAAAACTCCTGGCTGGTGGCCCATTTCCACAACGGTTTCTCCTACTGGATTTACGGTTGTATGG
GCGTTCTGGCAGCACTGTTTATGTGGAAATTTGTCCCGGAAACCAAAGGTAAAACCCTTGAGG
AGCTGGAAGCGCTCTGGGAACCGGAAACGAAGAAAACACAACAAACTGCTACGCTGCCAGA
AACTAAAGGTTTATCATTAGAAGAAGTTAATGATATGTACGCCGAAGGTGTTCTACCATGGAA
ATCAGCTTCCTGGGTTCCAGTATCCAAGAGAGGCGCTGACTACAACGCTGATGACCTAATGCA
TGATGACCAACCATTTTACAAGAGTTTGTTTAGCAGGAAATAA.

### REFERENCES

Beggs J D. Transformation of yeast by a replicating hybrid plasmid. Nature 1978; 275: 104-9.
Bessa D, Pereira F, Moreira R et al. Improved gap repair cloning in yeast:

treatment of the gapped vector with Taq DNA polymerase avoids vector self-ligation. Yeast 2012; 29: 419-23.
Broach J R, Strathern J N, Hicks J B. Transformation in yeast: Development of a hybrid cloning vector and isolation of the canl gene. Gene 1979; 8: 121-33.
Casal M, Paiva S, Andrade R P et al. The lactate-proton symport of Saccharomyces cerevisiae is encoded by JEN1. J Bacteriol 1999; 181: 2620-3.
Casal M, Queiros O,Talaia G et al. Carboxylic Acids Plasma Membrane Transporters in Saccharomyces cerevisiae. Adv Exp Med Biol 2016; 892: 229-51.
Cross B C S, Sinning I, Luirink J et al. Delivering proteins for export from the cytosol. Nat Rev Mol Cell Bio2009; 10: 255-64.
Davis E O, Henderson P J. The cloning and DNA sequence of the gene xylE for xylose-proton symport in Escherichia coli K12. Journal of Biological Chemistry 1987; 262: 13928-32.
Dobson L, Reményi I, Tusnády G E. CCTOP: a Consensus Constrained TOPology prediction web server. Nucleic Acids Res 2015; 43: W408-W12.
Fujita N, Nelson N, Fox T et al. Biosynthesis of the Torpedo californica acetylcholine receptor alpha subunit in yeast. Science 1986; 231: 1284-7.
Hinnen A, Hicks J B, Fink G R. Transformation of yeast. Proc Natl Acad Sci USA 1978; 75: 1929-33.
Lis P, Zarzycki M, Ko Y H et al. Transport and cytotoxicity of the anticancer drug 3-bromopyruvate in the yeast Saccharomyces cerevisiae. J Bioenerg Biomembr 2012; 44: 155-61.
Maier A, Volker B, Boles E et al. Characterisation of glucose transport in Saccharomyces cerevisiae with plasma membrane vesicles (countertransport) and intact cells (initial uptake) with single Hxt1, Hxt2, Hxt3, Hxt4, Hxt6, Hxt7 or Gal2 transporters. FEMS Yeast Res 2002; 2: 539-50.
McDermott J R, Rosen B P, Liu Z. Jen1p: a high affinity selenite transporter in yeast. Mol Biol Cell 2010; 21: 3934-41.
Mumberg D, Muller R, Funk M. Yeast vectors for the controlled expression of heterologous proteins in different genetic backgrounds. Gene 1995; 156: 119-22.
Núñez M F, Teresa Pellicer M, Badia J et al. The gene yghK linked to the glc operon of Escherichia coli encodes a permease for glycolate that is structurally and functionally similar to L-lactate permease. Microbiology 2001; 147: 1069-77.
Özcan S, Johnston M. Function and Regulation of Yeast Hexose Transporters. Microbiology and Molecular Biology Reviews 1999; 63: 554-69.
Pacheco A, Talaia G, Sa-Pessoa J et al. Lactic acid production in Saccharomyces cerevisiae is modulated by expression of the monocarboxylate transporters Jen1 and Ady2. FEMS Yeast Res 2012; 12: 375-81.
Paiva S, Kruckeberg A L, Casal M. Utilization of green fluorescent protein as a marker for studying the expression and turnover of the monocarboxylate permease Jen1p of Saccharomyces cerevisiae. Biochem J 2002; 363: 737-44.
Paiva S, Strachotova D, Kucerova H et al. The transport of carboxylic acids and important role of the Jen1p transporter during the development of yeast colonies. Biochem J 2013; 454: 551-8.
Queiros O, Pereira L, Paiva S et al. Functional analysis of Kluyveromyces lactic carboxylic acids permeases: heterologous expression of KUEN1 and KUEN2 genes. Curr Genet 2007; 51: 161-9.
Quistgaard E M, Löw C, Moberg P et al. Metal-mediated crystallization of the xylose transporter XylE from Escherichia coli in three different crystal forms. Journal of Structural Biology 2013; 184: 375-8.
Ribas D, Sá-Pessoa J, Soares-Silva I et al. Yeast as a tool to express sugar acid transporters with biotechnological interest. FEMS Yeast Res 2017; 17.
Soares-Silva I, Paiva S, Diallinas G et al. The conserved sequence NXX[S/T]HX[S/T]QDXXXT of the lactate/pyruvate:H(+) symporter subfamily defines the function of the substrate translocation pathway. Mol Membr Biol 2007; 24: 464-74.
Soares-Silva I, Ribas D, Foskolou I P et al. The Debaryomyces hansenii carboxylate transporters Jen1 homologues are functional in Saccharomyces cerevisiae. FEMS Yeast Res 2015; 15.
Sun L, Zeng X, Yan C et al. Crystal structure of a bacterial homologue of glucose transporters GLUT1-4. Nature 2012; 490: 361-6.
van Maris A J, Konings W N, van Dijken J P et al. Microbial export of lactic and 3-hydroxypropanoic acid: implications for industrial fermentation processes. Metab Eng 2004; 6: 245-55.
Wieczorke R, Krampe S, Weierstall T et al. Concurrent knock-out of at least 20 transporter genes is required to block uptake of hexoses in Saccharomyces cerevisiae. FEBS Letters 1999; 464: 123-8.
Young E, Poucher A, Cormer A et al. Functional Survey for Heterologous Sugar Transport Proteins, Using Saccharomyces cerevisiae as a Host. Appl Environ Microbiol 2011; 77: 3311-9.

1. A method for the production of a functional prokaryotic membrane transporter protein in a eukaryotic host organism comprising the following steps:
obtaining a DNA construct by ligating a DNA coding sequence of a prokaryotic membrane transporter protein to a N-terminal coding sequence and/or a C-terminal DNA coding sequence of a eukaryotic membrane protein;
introducing the obtained DNA construct in the eukaryotic host organism for the production of the functional prokaryotic membrane transporter protein.

2. The method according to claim 1, wherein the eukaryotic host organism is a fungus.

3. The method according to claim 2, wherein the fungus is a yeast.

4. The method according to claim 3, wherein the yeast is Saccharomyces cerevisiae.

5. The method according to claim 1, wherein the DNA coding sequence for the prokaryotic membrane transporter protein is from a prokaryotic cell.

6. The method according to claim 5, wherein the bacteria is Escherichia coli, Staphylococcus aureus, or combinations thereof.

7. The method according to claim 1, wherein the eukaryotic membrane protein is a membrane transporter protein.

8. The method according to claim 1, wherein the prokaryotic membrane transporter protein is a permease.

9. The method according to claim 8, wherein the permease is an organic acid permease, a sugar permease, or a mixture thereof.

10. The method according to claim 8, wherein the membrane transporter protein is a LldP lactate permease, a LctP lactate permease, a XylE xylose permease, or combinations thereof.

11. The method according to claim 1, wherein the DNA construct is obtained by ligating the DNA coding sequence for the prokaryotic membrane transporter protein between the N-terminal and the C-terminal DNA coding sequences of the eukaryotic membrane transporter protein.

12. The method according to claim 1, wherein the N-terminal coding DNA sequence is ligated before an initiation codon of the DNA coding sequence for the prokaryotic membrane transporter protein, and the C-terminal coding sequence is ligated after a penultimate codon of the DNA coding sequence for the prokaryotic membrane transporter protein.

13. The method according to claim 1, further comprising the separation and/or purification of the prokaryotic membrane transporter protein.

14. A DNA construct obtained by the method of claim 1, comprising a DNA coding sequence for a prokaryotic membrane transporter protein.

15. The DNA construct according to claim 14, wherein the DNA coding sequence for the prokaryotic membrane transporter protein is a permease, fused with the N-terminal and/or C-terminal DNA coding sequences of the eukaryotic membrane protein.

16. A eukaryotic host cell comprising the DNA construct of claim 15.

17. The eukaryotic host cell of claim 16, wherein the eukaryotic host cell increases cell transport capacity.

18. The eukaryotic host cell of 16, wherein the eukaryotic host cell increases the tolerance of eukaryotic organisms to intracellular compounds through the export of this molecule.


SEQUENCE LISTING (22 sequences)

SEQ ID NO: 1 - DNA, 1851 bp, Saccharomyces cerevisiae S288c
  JEN1
atgtcgtcgtcaattacagatgagaaaatatctggtgaacagcaacaacctgctggcagaaaactatactataacacaagtacatttgcagagcctcctctagtggacggagaaggtaaccctataaattatgagccggaagtttacaacccggatcacgaaaagctataccataacccatcactgcctgcacaatcaattcaggatacaagagatgatgaattgctggaaagagtttatagccaggatcaaggtgtagagtatgaggaagatgaagaggataagccaaacctaagcgctgcgtccattaaaagttatgctttaacgagatttacgtccttactgcacatccacgagttttcttgggagaatgtcaatcccatacccgaactgcgcaaaatgacatggcagaattggaactatttttttatgggttattttgcgtggttgtctgcggcttgggccttcttttgcgtttcagtatcagtcgctccattggctgaactatatgacagaccaaccaaggacatcacctgggggttgggattggtgttatttgttcgttcagcaggtgctgtcatatttggtttatggacagataagtcttccagaaagtggccgtacattacatgtttgttcttatttgtcattgcacaactctgtactccatggtgtgacacatacgagaaatttctgggcgtaaggtggataaccggtattgctatgggaggaatttacggatgtgcttctgcaacagcgattgaagatgcacctgtgaaagcacgttcgttcctatcaggtctatttttttctgcttacgctatggggttcatatttgctatcattttttacagagcctttggctactttagggatgatggctggaaaatattgttttggtttagtatttttctaccaattctactaattttctggagattgttatggcctgaaacgaaatacttcaccaaggttttgaaagcccgtaaattaatattgagtgacgcagtgaaagctaatggtggcgagcctctaccaaaagccaactttaaacaaaagatggtatccatgaagagaacagttcaaaagtactggttgttgttcgcatatttggttgttttattggtgggtccaaattacttgactcatgcttctcaagacttgttgccaaccatgctgcgtgcccaattaggcctatccaaggatgctgtcactgtcattgtagtggttaccaacatcggtgctatttgtgggggtatgatatttggacagttcatggaagttactggaagaagattaggcctattgattgcatgcacaatgggtggttgcttcacctaccctgcatttatgttgagaagcgaaaaggctatattaggtgccggtttcatgttatatttttgtgtctttggtgtctggggtatcctgcccattcaccttgcagagttggcccctgctgatgcaagggctttggttgccggtttatcttaccagctaggtaatctagcttctgcagcggcttccacgattgagacacagttagctgatagatacccattagaaagagatgcctctggtgctgtgattaaagaagattatgccaaagttatggctatcttgactggttctgttttcatcttcacatttgcttgtgtttttgttggccatgagaaattccatcgtgatttgtcctctcctgttatgaagaaatatataaaccaagtggaagaatacgaagccgatggtctttcgattagtgacattgttgaacaaaagacggaatgtgcttcagtgaagatgattgattcgaacgtctcaaagacatatgaggagcatattgagaccgtttaa

SEQ ID NO: 2 - DNA, 1713 bp, Saccharomyces cerevisiae S288c
  HXT1
atgaattcaactcccgatctaatatctcctcagaaatccaattcatccaactcatatgaattggaatctggtcgttcaaaggccatgaatactccagaaggtaaaaatgaaagttttcacgacaacttaagtgaaagtcaagtgcaacccgccgttgcccctccaaacaccggaaaaggtgtctacgtaacggtttctatctgttgtgttatggttgctttcggtggtttcatatttggatgggatactggtaccatttctggttttgttgctcaaactgattttctaagaagatttggtatgaagcaccacgacggtagtcattacttgtccaaggtgagaactggtttaattgtctctatttttaacattggttgtgccattggtggtatcgtcttagccaagctaggtgatatgtatggtcgtagaatcggtttgattgtcgttgtagtaatctacactatcggtatcattattcaaatagcctcgatcaacaagtggtaccaatatttcattggtagaattatctctggtttaggtgtcggtggtatcacagttttatctcccatgctaatatctgaggtcgcccccagtgaaatgagaggcaccttggtttcatgttaccaagtcatgattactttaggtattttcttaggttactgtaccaattttggtaccaagaattactcaaactctgtccaatggagagttccattaggtttgtgtttcgcctgggccttatttatgattggtggtatgatgtttgttcctgaatctccacgttatttggttgaagctggcagaatcgacgaagccagggcttctttagctaaagttaacaaatgcccacctgaccatccatacattcaatatgagttggaaactatcgaagccagtgtcgaagaaatgagagccgctggtactgcatcttggggcgaattattcactggtaaaccagccatgtttcaacgtactatgatgggtatcatgattcaatctctacaacaattaactggtgataactatttcttctactacggtaccattgttttccaggctgtcggtttaagtgactcttttgaaacttctattgtctttggtgtcgtcaacttcttctccacttgttgttctctgtacaccgttgaccgttttggccgtcgtaactgtttgatgtggggtgctgtcggtatggtctgctgttatgttgtctatgcctctgttggtgttaccagattatggccaaacggtcaagatcaaccatcttcaaagggtgctggtaactgtatgattgttttcgcatgtttctacattttctgtttcgctactacctgggccccaattgcttacgttgttatttcagaatgtttcccattaagagtcaaatccaagtgtatgtctattgccagtgctgctaactggatctggggtttcttgattagtttcttcaccccatttattactggtgccatcaacttctactacggttacgttttcatgggctgtatggttttcgcttacttttacgtctttttcttcgttccagaaactaaaggtttatcattagaagaagttaatgatatgtacgccgaaggtgttctaccatggaaatcagcttcctgggttccagtatccaagagaggcgctgactacaacgctgatgacctaatgcatgatgaccaaccattttacaagagtttgtttagcaggaaataa

SEQ ID NO: 3 - DNA, 1656 bp, Escherichia coli str. K-12 substr. MG1655
  lldP
atgaatctctggcaacaaaactacgatcccgccgggaatatctggctttccagtctgatagcatcgcttcccatcctgtttttcttctttgcgctgattaagctcaaactgaaaggatacgtcgccgcctcgtggacggtggcaatcgcccttgccgtggctttgctgttctataaaatgccggtcgctaacgcgctggcctcggtggtttatggtttcttctacgggttgtggcccatcgcgtggatcattattgcagcggtgttcgtctataagatctcggtgaaaaccgggcagtttgacatcattcgctcgtctattctttcgataacccctgaccagcgtctgcaaatgctgatcgtcggtttctgtttcggcgcgttccttgaaggagccgcaggctttggcgcaccggtagcaattaccgccgcattgctggtcggcctgggttttaaaccgctgtacgccgccgggctgtgcctgattgttaacaccgcgccagtggcatttggtgcgatgggcattccaatcctggttgccggacaggtaacaggtatcgacagctttgagattggtcagatggtggggcggcagctaccgtttatgaccattatcgtgctgttctggatcatggcgattatggacggctggcgcggtatcaaagagacgtggcctgcggtcgtggttgcgggcggctcgtttgccatcgctcagtaccttagctctaacttcattgggccggagctgccggacattatctcttcgctggtatcactgctctgcctgacgctgttcctcaaacgctggcagccagtgcgtgtattccgttttggtgatttgggggcgtcacaggttgatatgacgctggcccacaccggttacactgcgggtcaggtgttacgtgcctggacaccgttcctgttcctgacagctaccgtaacactgtggagtatcccgccgtttaaagccctgttcgcatcgggtggcgcgctgtatgagtgggtgatcaatattccggtgccgtacctcgataaactggttgcccgtatgccgccagtggtcagcgaggctacagcctatgccgccgtgtttaagtttgactggttctctgccaccggcaccgccattctgtttgctgcactgctctcgattgtctggctgaagatgaaaccgtctgacgctatcagcaccttcggcagcacgctgaaagaactggctctgcccatctactccatcggtatggtgctggcattcgcctttatttcgaactattccggactgtcatcaacactggcgctggcactggcgcacaccggtcatgcattcaccttcttctcgccgttcctcggctggctgggggtattcctgaccgggtcggatacctcatctaacgccctgttcgccgcgctgcaagccaccgcagcacaacaaattggcgtctctgatctgttgctggttgccgccaataccaccggtggcgtcaccggtaagatgatctccccgcaatctatcgctatcgcctgtgcggcggtaggcctggtgggcaaagagtctgatttgttccgctttactgtcaaacacagcctgatcttcacctgtatagtgggcgtgatcaccacgcttcaggcttatgtcttaacgtggatgattccttaa

SEQ ID NO: 4 - DNA, 1593 bp, Staphylococcus aureus subsp. aureus NCTC 8325
  lctP
atgacactacttactgtaaatccattcgataatgtcggattatcagccttagttgcagcagtacctattattttatttttattatgcttaaccgtttttaaaatgaaaggcatttatgcagcattgacaactttggttgttacattgattgtggctttatttgtatttgaattaccagcgcgtgtatcagcaggtgcgattacagaaggcgttgttgccggtattttcccaataggatatatcgttttaatggcagtttggttatataaagtttctattaaaacaggacaattttctattattcaagatagtattgcaagtatttcagtggaccaaagaatccaactattattaattggattttgtttcaacgcatttttagaaggtgcagcaggatttggtgtgccaattgcgatttgtgcagtattattaattcaacttggatttgaaccattaaaagcagcgatgttatgtttaattgctaatggtgcggcgggtgcctttggtgcaattggtttaccagttagtattattgatacgtttaacttaagtggaggcgttacaacattagatgttgcgagatactcagcattaacacttccaattttaaactttattattccatttgttttagtattcattgtagatggtatgaaaggtattaaagaaattttacctgtcattttaacagtgagtggtacatatactggattacaattattattaacaatattccatggtccagaactagcagacattattccatcactagcaacaatggtggtgttagcatttgtttgtcgtaaatttaaaccgaaaaacattttcagattggaagcgtctgaacataaaattcaaaaacgaacgcctaaagaaattgtctttgcttggagtccgttcgtaattttaactgcctttgtattagtatggagtgcaccattcttcaaaaaattattccaacctggaggtgcacttgaaagtttagtaataaaattgccaattccaaatactgtgagtgatttatcgcctaaaggaattgcgttgcgtctcgatttaattggtgcaactgggacagcgattttattaacagtaattattacaattttaattacgaagttaaaatggaaaagtgcaggtgctttattggtcgaagcaattaaagaattatggttaccgatccttacaatttcagctatcctagctattgctaaagttatgacatacggtggtttgactgtagcaattggacaaggtattgctaaagcgggagcaattttcccattattctctccagtattaggttggattggtgtgtttatgactggttcagttgtaaataacaatactttattcgcacctattcaagcgacagtggcacaacaaatttcaacaagcggttcattacttgtggcagctaacactgcaggtggtgtagcagcgaaacttatttcaccacaatcaattgccattgcgactgcagctgttaaaaaagttggtgaagaatctgcattattaaaaatgacgttaaaatacagtattatatttgttgcttttatttgtgtttggacgtttatactaacgttaatattctaa

SEQ ID NO: 5 - DNA, 1473 bp, Artificial Sequence
  xylE
atgaatacacaatacaactcttcatacattttctctatcactttggttgctacattaggtggtttgttgttcggttacgatactgcagttatttctggtacagttgaatcattgaacactgttttcgttgctccacaaaatttgtctgaatcagctgcaaattctttgttaggtttttgtgttgcttcagcattgattggttgtattattggtggtgcattaggtggttactgttctaacagattcggtagaagagattcattgaagatcgctgcagttttgtttttcatctctggtgttggttcagcttggccagaattgggttttacatctattaatccagataacactgttccagtttatttggcaggttacgttccagaattcgttatctatagaatcatcggtggtattggtgttggtttggcttctatgttatcaccaatgtacattgcagaattggctccagcacatattcgtggtaaattggtttcttttaatcaattcgctatcatcttcggtcaattgttagtttattgtgttaattactttattgctagatctggtgacgcatcatggttgaatactgacggctggcgttatatgtttgcctcggaatgtatccctgcactgctgttcttaatgctgctgtataccgtgccagaaagtcctcgctggctgatgtcgcgcggcaagcaagaacaggcggaaggtatcctgcgcaaaattatgggcaacacgcttgcaactcaggcagtacaggaaattaaacactccctggatcatggccgcaaaaccggtggtcgtctgctgatgtttggcgtgggcgtgattgtaatcggcgtaatgctctccatcttccagcaatttgtcggcatcaatgtggtgctgtactacgcgccggaagtgttcaaaacgctgggggccagcacggatatcgcgctgttgcagaccattattgtcggagttatcaacctcaccttcaccgttctggcaattatgacggtggataaatttggtcgtaagccactgcaaattatcggcgcactcggaatggcaatcggtatgtttagcctcggtaccgcgttttacactcaggcaccgggtattgtggcgctactgtcgatgctgttctatgttgccgcctttgccatgtcctggggtccggtatgctgggtactgctgtcggaaatcttcccgaatgctattcgtggtaaagcgctggcaatcgcggtggcggcccagtggctggcgaactacttcgtctcctggaccttcccgatgatggacaaaaactcctggctggtggcccatttccacaacggtttctcctactggatttacggttgtatgggcgttctggcagcactgtttatgtggaaatttgtcccggaaaccaaaggtaaaacccttgaggagctggaagcgctctgggaaccggaaacgaagaaaacacaacaaactgctacgctg

SEQ ID NO: 6 - DNA, 3030 bp, Artificial Sequence
  NJ-LldP-CJ-GFP
atgtcgtcgtcaattacagatgagaaaatatctggtgaacagcaacaacctgctggcagaaaactatactataacacaagtacatttgcagagcctcctctagtggacgaagaaggtaaccctataaattatgagccggaagtttacaacccggatcacgaaaagctataccataacccatcactgcctgcacaatcaattcaggatacaagagatgatgaattgctggaaagagtttatagccaggatcaaggtgtagagtatgaggaagatgaagaggataagccaaacctaagcgctgcgtccattaaaagttatgctttaacgagatttacgtccttactgcacatccacgagttttcttgggagaatgtcaatcccatacccgaactgcgcaaaatgacatggcagaattggaactatatgaatctctggcaacaaaactacgatcccgccgggaatatctggctttccagtctgatagcatcgcttcccatcctgtttttcttctttgcgctgattaagctcaaactgaaaggatacgtcgccgcctcgtggacggtggcaatcgcccttgccgtggctttgctgttctataaaatgccggtcgctaacgcgctggcctcggtggtttatggtttcttctacgggttgtggcccatcgcgtggatcattattgcagcggtgttcgtctataagatctcggtgaaaaccgggcagtttgacatcattcgctcgtctattctttcgataacccctgaccagcgtctgcaaatgctgatcgtcggtttctgtttcggcgcgttccttgaaggagccgcaggctttggcgcaccggtagcaattaccgccgcattgctggtcggcctgggttttaaaccgctgtacgccgccgggctgtgcctgattgttaacaccgcgccagtggcatttggtgcgatgggcattccaatcctggttgccggacaggtaacaggtatcgacagctttgagattggtcagatggtggggcggcagctaccgtttatgaccattatcgtgctgttctggatcatggcgattatggacggctggcgcggtatcaaagagacgtggcctgcggtcgtggttgcgggcggctcgtttgccatcgctcagtaccttagctctaacttcattgggccggagctgccggacattatctcttcgctggtatcactgctctgcctgacgctgttcctcaaacgctggcagccagtgcgtgtattccgttttggtgatttgggggcgtcacaggttgatatgacgctggcccacaccggttacactgcgggtcaggtgttacgtgcctggacaccgttcctgttcctgacagctaccgtaacactgtggagtatcccgccgtttaaagccctgttcgcatcgggtggcgcgctgtatgagtgggtgatcaatattccggtgccgtacctcgataaactggttgcccgtatgccgccagtggtcagcgaggctacagcctatgccgccgtgtttaagtttgactggttctctgccaccggcaccgccattctgtttgctgcactgctctcgattgtctggctgaagatgaaaccgtctgacgctatcagcaccttcggcagcacgctgaaagaactggctctgcccatctactccatcggtatggtgctggcattcgcctttatttcgaactattccggactgtcatcaacactggcgctggcactggcgcacaccggtcatgcattcaccttcttctcgccgttcctcggctggctgggggtattcctgaccgggtcggatacctcatctaacgccctgttcgccgcgctgcaagccaccgcagcacaacaaattggcgtctctgatctgttgctggttgccgccaataccaccggtggcgtcaccggtaagatgatctccccgcaatctatcgctatcgcctgtgcggcggtaggcctggtgggcaaagagtctgatttgttccgctttactgtcaaacacagcctgatcttcacctgtatagtgggcgtgatcaccacgcttcaggcttatgtcttaacgtggatgattcctatggctatcttgactggttctgttttcatcttcacatttgcttgtgtttttgttggccatgagaaattccatcgtgatttgtcctctcctgttatgaagaaatatataaaccaagtggaagaatacgaagccgatggtctttcgattagtgacattgttgaacaaaagacggaatgtgcttcagtgaagatgattgattcgaacgtctcaaagacatatgaggagcatattgagaccgttagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatag

SEQ ID NO: 7 - DNA, 2968 bp, Artificial Sequence
  NJ-LctP-CJ-GFP
atgtcgtcgtcaattacagatgagaaaatatctggtgaacagcaacaacctgctggcagaaaactatactataacacaagtacatttgcagagcctcctctagtggacgaagaaggtaaccctataaattatgagccggaagtttacaacccggatcacgaaaagctataccataacccatcactgcctgcacaatcaattcaggatacaagagatgatgaattgctggaaagagtttatagccaggatcaaggtgtagagtatgaggaagatgaagaggataagccaaacctaagcgctgcgtccattaaaagttatgctttaacgagatttacgtccttactgcacatccacgagttttcttgggagaatgtcaatcccatacccgaactgcgcaaaatgacatggcagaattggaactatatgacactacttactgtaaatccattcgataatgtcggattatcagccttagttgcagcagtacctattattttatttttattatgcttaaccgtttttaaaatgaaaggcatttatgcagcattgacaactttggttgttacattgattgtggctttatttgtatttgaattaccagcgcgtgtatcagcaggtgcgattacagaaggcgttgttgccggtattttcccaataggatatatcgttttaatggcagtttggttatataaagtttctattaaaacaggacaattttctattattcaagatagtattgcaagtatttcagtggaccaaagaatccaactattattaattggattttgtttcaacgcatttttagaaggtgcagcaggatttggtgtgccaattgcgatttgtgcagtattattaattcaacttggatttgaaccattaaaagcagcgatgttatgtttaattgctaatggtgcggcgggtgcctttggtgcaattggtttaccagttagtattattgatacgtttaacttaagtggaggcgttacaacattagatgttgcgagatactcagcattaacacttccaattttaaactttattattccatttgttttagtattcattgtagatggtatgaaaggtattaaagaaattttacctgtcattttaacagtgagtggtacatatactggattacaattattattaacaatattccatggtccagaactagcagacattattccatcactagcaacaatggtggtgttagcatttgtttgtcgtaaatttaaaccgaaaaacattttcagattggaagcgtctgaacataaaattcaaaaacgaacgcctaaagaaattgtctttgcttggagtccgttcgtaattttaactgcctttgtattagtatggagtgcaccattcttcaaaaaattattccaacctggaggtgcacttgaaagtttagtaataaaattgccaattccaaatactgtgagtgatttatcgcctaaaggaattgcgttgcgtctcgatttaattggtgcaactgggacagcgattttattaacagtaattattacaattttaattacgaagttaaaatggaaaagtgcaggtgctttattggtcgaagcaattaaagaattatggttaccgatccttacaatttcagctatcctagctattgctaaagttatgacatacggtggtttgactgtagcaattggacaaggtattgctaaagcgggagcaattttcccattattctctccagtattaggttggattggtgtgtttatgactggttcagttgtaaataacaatactttattcgcacctattcaagcgacagtggcacaacaaatttcaacaagcggttcattacttgtggcagctaacactgcaggtggtgtagcagcgaaacttatttcaccacaatcaattgccattgcgactgcagctgttaaaaaagttggtgaagaatctgcattattaaaaatgacgttaaaatacagtattatatttgttgcttttatttgtgtttggacgtttatactaacgttaatattctatggctatcttgactggttctgttttcatcttcacatttgcttgtgtttttgttggccatgagaaattccatcgtgatttgtcctctcctgttatgaagaaatatataaaccaagtggaagaatacgaagccgatggtctttcgattagtgacattgttgaacaaaagacggaatgtgcttcagtgaagatgattgattcgaacgtctcaaagacatatgaggagcatattgagaccgttagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatag

SEQ ID NO: 8 - DNA, 1824 bp, Artificial Sequence
  NH-XylE-CH
atgaattcaactcccgatctaatatctcctcagaaatccaattcatccaactcatatgaattggaatctggtcgttcaaaggccatgaatactccagaaggtaaaaatgaaagttttcacgacaacttaagtgaaagtcaagtgcaacccgccgttgcccctccaaacaccggaaaaatgaatacacaatacaactcttcatacattttctctatcactttggttgctacattaggtggtttgttgttcggttacgatactgcagttatttctggtacagttgaatcattgaacactgttttcgttgctccacaaaatttgtctgaatcagctgcaaattctttgttaggtttttgtgttgcttcagcattgattggttgtattattggtggtgcattaggtggttactgttctaacagattcggtagaagagattcattgaagatcgctgcagttttgtttttcatctctggtgttggttcagcttggccagaattgggttttacatctattaatccagataacactgttccagtttatttggcaggttacgttccagaattcgttatctatagaatcatcggtggtattggtgttggtttggcttctatgttatcaccaatgtacattgcagaattggctccagcacatattcgtggtaaattggtttcttttaatcaattcgctatcatcttcggtcaattgttagtttattgtgttaattactttattgctagatctggtgacgcatcatggttgaatactgacggctggcgttatatgtttgcctcggaatgtatccctgcactgctgttcttaatgctgctgtataccgtgccagaaagtcctcgctggctgatgtcgcgcggcaagcaagaacaggcggaaggtatcctgcgcaaaattatgggcaacacgcttgcaactcaggcagtacaggaaattaaacactccctggatcatggccgcaaaaccggtggtcgtctgctgatgtttggcgtgggcgtgattgtaatcggcgtaatgctctccatcttccagcaatttgtcggcatcaatgtggtgctgtactacgcgccggaagtgttcaaaacgctgggggccagcacggatatcgcgctgttgcagaccattattgtcggagttatcaacctcaccttcaccgttctggcaattatgacggtggataaatttggtcgtaagccactgcaaattatcggcgcactcggaatggcaatcggtatgtttagcctcggtaccgcgttttacactcaggcaccgggtattgtggcgctactgtcgatgctgttctatgttgccgcctttgccatgtcctggggtccggtatgctgggtactgctgtcggaaatcttcccgaatgctattcgtggtaaagcgctggcaatcgcggtggcggcccagtggctggcgaactacttcgtctcctggaccttcccgatgatggacaaaaactcctggctggtggcccatttccacaacggtttctcctactggatttacggttgtatgggcgttctggcagcactgtttatgtggaaatttgtcccggaaaccaaaggtaaaacccttgaggagctggaagcgctctgggaaccggaaacgaagaaaacacaacaaactgctacgctgccagaaactaaaggtttatcattagaagaagttaatgatatgtacgccgaaggtgttctaccatggaaatcagcttcctgggttccagtatccaagagaggcgctgactacaacgctgatgacctaatgcatgatgaccaaccattttacaagagtttgtttagcaggaaataa

SEQ ID NO: 9 - DNA, 53 bp, Artificial Sequence
  Ld_1
aactgcgcaaaatgacatggcagaattggaactatatgaatctctggcaacaa

SEQ ID NO: 10 - DNA, 50 bp, Artificial Sequence
  Ld_2
aatgtgaagatgaaaacagaaccagtcaagatagcaggaatcatccacgt

SEQ ID NO: 11 - DNA, 53 bp, Artificial Sequence
  Lc_1
aactgcgcaaaatgacatggcagaattggaactatatgacactacttactgta

SEQ ID NO: 12 - DNA, 52 bp, Artificial Sequence
  Lc_2
aatgtgaagatgaaaacagaaccagtcaagatagcgaatattaacgttagta

SEQ ID NO: 13 - DNA, 52 bp, Artificial Sequence
  XylE1
cccgccgttgcccctccaaacaccggaaaaatgaatacacaatacaactctt

SEQ ID NO: 14 - DNA, 47 bp, Artificial Sequence
  XylE2
acttcttctaatgataaacctttagtttctggcagcgtagcagtttg

SEQ ID NO: 15 - DNA, 27 bp, Artificial Sequence
  LlFWD
gggggatccatgaatctctggcaacaa

SEQ ID NO: 16 - DNA, 27 bp, Artificial Sequence
  LlREV
ggggaattcttaaggaatcatccacgt

SEQ ID NO: 17 - DNA, 27 bp, Artificial Sequence
  LcFWD
gggggatccatgacactacttactgta

SEQ ID NO: 18 - DNA, 29 bp, Artificial Sequence
  LcREV
ggggaattcttagaatattaacgttagta

SEQ ID NO: 19 - DNA, 49 bp, Artificial Sequence
  HF
gatcccccgggctgcaggaattcgatatcaatgaattcaactcccgatc

SEQ ID NO: 20 - DNA, 53 bp, Artificial Sequence
  HR
catgactcgaggtcgacggtatcgataagctttatttcctgctaaacaaactc

SEQ ID NO: 21 - DNA, 48 bp, Artificial Sequence
  Hxt1F
gacaactccagtgaaaagttcttctcctttacttttcctgctaaacaa

SEQ ID NO: 22 - DNA, 67 bp, Artificial Sequence
  GFPR
ttacatgactcgaggtcgacggtatcgataagcttgatatcgaactatttgtatagttcatccatgc